(19) United States
(12) Patent Application Publication
(10) Pub. No.: US 2018/0242917 A1
(43) Pub. Date: Aug. 30, 2018

(54) SYSTEMS AND METHODS FOR DETERMINING IMAGE QUALITY AND MODELS BASED ON CONTRAST INJECTION PROTOCOLS

(71) Applicant: Duke University, Durham, NC (US)

(72) Inventors: Pooyan Sahbaee Bagherzadeh, Durham, NC (US); Ehsan Samei, Durham, NC (US); Paul Segars, Durham, NC (US)

(21) Appl. No.: 15/753,988

(22) PCT Filed: Aug. 22, 2016
(86) PCT No.: PCT/US16/48090

§ 371 Date: Feb. 21, 2018

Related U.S. Application Data
(60) Provisional application No. 62/208,013, filed on Aug. 21, 2015.
(60) Provisional application No. 62/208,026, filed on Aug. 21, 2015.

(51) Int. Cl. A61B 5/00; A61B 5/026; A61B 5/0295; A61B 6/03; A61B 6/00; A61M 5/00; G01R 33/56; G01R 33/563; G06T 7/00

(52) U.S. Cl. CPC A61B 5/7221; G06T 2207/10088; A61B 5/0295; A61B 6/032; A61B 6/504; A61B 6/507; A61M 5/007; A61B 5/7203; A61B 6/5294; A61B 6/5258; G01R 33/5601; G01R 33/56366; G01R 33/5608; G06T 7/0012; G06T 2207/10081; A61B 5/0263

(57) ABSTRACT

Systems and methods for determining image quality and models based on contrast injection protocols. According to an aspect, a method includes providing a contrast material injection protocol for imaging of a target portion of a subject. The method also includes determining a model of propagation of contrast material through the target portion. Further, the method includes determining quality of imaging of the target portion based on the contrast material injection protocol and model.

### CROSS REFERENCE TO RELATED APPLICATIONS

This application claims priority to U.S. Provisional Patent Application No. 62/208,013, filed Aug. 21, 2015, and U.S. Provisional Patent Application No. 62/208,026, filed Aug. 21, 2015; the disclosures of which are incorporated herein by reference in their entireties.

### FEDERALLY SPONSORED RESEARCH OR DEVELOPMENT

This invention was made with government support under grant 2R01EB001838-09A1 awarded by National Institutes of Health (NIH). The government has certain rights to the invention.

### TECHNICAL FIELD

The present disclosure relates to imaging. More particularly, the present disclosure relates to systems and methods for determining image quality and models based on contrast injection protocols.

### BACKGROUND

A CT scan makes use of computer-processed combinations of X-ray images captured from different angles to produce cross-sectional (tomographic) images of specific areas of a scanned object. Medical imaging is a common application of X-ray CT. Its cross-sectional images are used for diagnostic and therapeutic purposes in various medical disciplines. Another imaging system often utilized is MRI, which is a technique used in radiology to image a subject. MRI uses strong magnetic fields, radio waves, and field gradients to form images of the subject.

The technological advances in CT conveying the high quality tomography and improved diagnosis, has led to phenomenal growth and widespread acceptance in medical imaging. Presently, CT is the largest man-made source of medical radiation exposure, constituting half of the total exposure to the United States population. Exposure is inherently related to contrast injection. In clinical practice the administration of contrast material is widely used to improve the image quality and diagnostic sensitivity. Currently, over 60% of total CT examination routines performed in United States are contrast enhanced. However, there is a variable implementation across institutions, i.e. in some institution, patients with different size and age receive the same amount of contrast agent and undergo the identical scan procedure, while in some institution the amount of contrast is adjusted based on patient size and age. Overall, there is not a consistent systematic administration technique across the clinical practice. Consequently, the current state of practice for contrast material administration leads to inconsistent enhancement across different patients.

In addition, it is noted that CT imaging, although resulting in improvements in patient diagnosis and treatment, has raised new challenges and concerns related to the population risk from radiation exposure. To promise the most effective and safe utilization of imaging technologies, optimization in the use of medical systems is more essential than ever. Clinical trials offer the most representative reflection of a system performance. However, clinical trials are cumbersome, costly, and unpractical for a multiplicity of optimization options. Virtual clinical trials (VCTs), which involve the use of computational phantoms and models of the imaging process, can offer a more efficient means to evaluate current and emerging imaging systems and methods. Such trials are being increasingly explored for imaging research.

VCTs require a realistic computerized patient population to serve as the known truth. A myriad of computerized human phantoms have been developed that can be used for VCT. However, most of these phantoms are limited due to oversimplified stylized structures or being representative of only a limited number of patient's body sizes. To address these limitations, libraries of computerized 4D extended cardiac-torso (XCAT) human models based on real CT images encompassing diverse anatomies and patient's body sizes have been developed. While effective for a wide range of medical imaging studies, the current XCAT models suffer from a major drawback, i.e., the lack of modeling of the vascular and parenchymal enhancement that occurs during contrast-enhanced CT scans. This can severely limit the XCAT in its abilities to conduct virtual studies for evaluating and optimizing current CT imaging technologies.

In view of the foregoing, there is a need for techniques and systems for improved CT and MRI planning and imaging.

### SUMMARY

Disclosed herein are systems and methods for determining CT and MRI image quality and models based on contrast injection protocols. Systems and methods disclosed herein can incorporate dynamics of blood flow and thus the distribution of contrast material or medium into an anthropomorphic phantom to generate a four-dimensional (4D) patient model for multimodality imaging studies.

Further, systems and methods disclosed herein can facilitate the design and optimization of new contrast material administration techniques. Such techniques can be orchestrated with scan parameters (e.g. scan timing) based on a relationship between contrast enhancement and the injection function. Knowing the dynamics of contrast material distribution, systems and methods disclosed herein can be used for modeling the injection protocols for different patients based on the desired contrast enhancements. Such systems and methods can provide for an integrated optimization of contrast medium dose, radiation dose, image quality, and patient-specific contrast administration protocols. Further, systems and methods disclosed herein can be used to determine the required contrast material injection function to achieve a desired contrast enhancement in specific organs and vessels.

According to an aspect, a method includes providing a contrast material injection protocol for imaging of a target portion of a subject. The method also includes determining a model of propagation of contrast material through the target portion. Further, the method includes determining quality of imaging of the target portion based on the contrast material injection protocol and the model.

According to another aspect, a method includes providing a model of propagation of contrast material through a target portion of a subject. Further, the method includes determining different contrast material injection protocols for imaging of the target portion of the subject. The method also includes determining, based on the model, one of the contrast material injection protocols that optimizes image quality of the target.

According to another aspect, a method includes providing a model of propagation of contrast material through a target portion of a subject. The method also includes determining different contrast material injection protocols for imaging of the target portion of the subject. Further, the method includes determining, based on the model, one of the contrast material injection protocols that optimizes contrast material dose during imaging of the target portion.

According to another aspect, a method includes providing a model of propagation of contrast material through a target portion of subject. The method also includes determining different contrast material injection protocols for imaging of the target portion of the subject. Further, the method includes determining, based on the model, one of the contrast material injection protocols that optimizes radiation dose during imaging of the target portion.

According to another aspect, a method includes determining one of a desired quality and radiation dose of imaging of a target portion of a subject. The method also includes determining a model of propagation of contrast material through the target portion. Further, the method includes determining, based on the model, a contrast material injection protocol to achieve the desired one of quality and radiation dose of imaging of the target.

### BRIEF DESCRIPTION OF THE DRAWINGS

The summary above, as well as the following detailed description of illustrative embodiments, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following figures.

FIG. 1 is a schematic diagram of an example CT imaging system for estimating irradiation dose for patient application based on a radiation field model and a patient anatomy model in accordance with embodiments of the present disclosure.

FIG. 2 illustrates a diagram of a physiologically based pharmacokinetic (PBPK) compartmental model for simulating distribution pharmacokinetics of contrast medium or material via the cardiovascular system through the body.

FIGS. 3A and 3B show enhancement curves calculated in kidney applying the analytical inverse model for (a) a uni-phasic (injection of 125 mL of contrast medium of 320 mgI/ml iodine concentration at 5 mL/s) and (b) a bi-phasic (injection of 50 mL of the same contrast medium at 2.5 mL/sec followed by 75 mL at 1 mL/sec) injection protocol, respectively.

FIG. 4 shows an error map created for kidney. The time-to-peak, peak value, and mean absolute errors in prediction of the contrast enhancement in kidney based on predicted injection functions applying the analytical inverse model across a) uni-phasic and b) bi-phasic injection protocols. Each block includes 16 different combinations of 4 iodine concentrations (y-axis) and 4 injection durations (x-axis).

FIG. 5 shows error bars created for different organs. The maximum value of errors in prediction of the contrast enhancement based on predicted injection functions applying analytical inverse method across uni-phasic and bi-phasic injection protocols are shown for a) aorta, b) kidney, c) stomach, and d) small intestine.

FIG. 6 shows (a) a coronal image of XCAT phantom, and (b) radiation dose to the liver at different phases (pre-contrast, arterial, portal, and delay phase) of scan for a reference XCAT adult male model (30 y.o., 75 kg, 170 cm) “undergoing” a liver contrast enhanced CT scan with injection of 120 mL of contrast medium (350 mgI/mL) at 4 mL/s.

FIG. 7 depicts a PBPK compartmental model used for simulating pharmacokinetics of contrast medium via the cardiovascular system through the body. The model includes 38 compartments: each ellipse represents a vessel compartment and each box represents an organ (see Table I).

FIG. 8 depicts diagrams of (a) organ and (b) vessel compartments. Each organ is modeled by 3 sub-compartments: intravascular, extracellular, and intracellular; and are intravascular and extracellular concentrations; and are intravascular and extravascular volumes. Note that the input and output blood flow rates (Q) are assumed to be equal.

FIG. 9 is an image of an example collage of extended cardiac (XCAT) patient phantom with variety of age, size, gender, and anatomical diversity used in a study.

FIG. 10 shows graphs of intravenous contrast agent injection protocols. (Left) a uni-phasic injection of 125 mL of contrast material (320 mgI/mL) at 5 mL/sec and (Right) a bi-phastic injection of 50 mL of the same contrast agent at 2.5 mL/sec followed by 75 mL at 1 mL/sec.

FIG. 11 shows images of distribution of contrast material throughout a 5D XCAT model undergoing a uni-phastic injection protocol of 125 mL of contrast agent (320 mgI/mL) at 5 mL/sec in the first 100 seconds after the injection.

FIG. 12 is a graph of iodine concentration curves for different organs (brain, heart, kidney, lung, liver, pancreas, small intestine, and stomach) used to update the particular organ's material as a function of time in a male XCAT model.

FIG. 13 shows graphs of simulated and clinical aortic and hepatic contrast enhancement-time curves with uni and bi-phasic injections. Simulated results are shown with thin solid curves for the aorta and liver. The thicker black curves represent the mean value of simulation data for each data set.

FIG. 14 depicts simulated contrast enhancement curves for different organs (brain, heart, kidney, lung, liver, pancreas, small intestine, and stomach) across a library of 5D XCAT models undergoing a uni-phasic injection of 125 mL of contrast agent (320 mgI/mL) at 5 mL/sec.

FIG. 15 depicts contrast enhancement curves for different organs (brain, heart, kidney, lung, liver, pancreas, small intestine, and stomach) across a library of 5D XCAT models undergoing a bi-phasic injection of 50 mL of the same contrast agent at 2.5 mL/sec followed by 75 mL at 1 mL/sec.

FIG. 16 is a screen display of a contrast enhancement calculator in accordance with embodiments of the present disclosure.

FIG. 17 illustrates a method of an example method for determining image quality in accordance with embodiments of the present disclosure.

FIG. 18 illustrates a method of an example method for determining a contrast material injection protocol that optimizes image quality of a target portion of a subject in accordance with embodiments of the present disclosure.

FIG. 19 illustrates a method of an example method for determining a contrast material injection protocol that optimizes contrast material dose during imaging of a target portion of a subject in accordance with embodiments of the present disclosure.

FIG. 20 illustrates a method of an example method for determining a contrast material injection protocol that optimizes radiation dose during imaging of a target portion of a subject in accordance with embodiments of the present disclosure.

FIG. 21 illustrates a method of an example method for a contrast material injection protocol to achieve a desired quality or radiation dose of imaging of a target of a subject in accordance with embodiments of the present disclosure.

FIG. 22 illustrates graphs showing an example iterative stripping model in accordance with embodiments of the present disclosure. This figure summarizes example steps of the iterative stripping technique. The unity signal strip, adjusted signal strip, and predicted uniphasic injection function are shown at the top of the figure. The reference response, adjusted response functions, desired IC (calculated from the contrast enhancement), and subtracted reference functions are shown at the bottom of the figure.

FIGS. 23A and 23B depict an error map created for kidneys. The time-to-peak, peak value, and MEAs in the prediction of the contrast enhancement in the kidney are based on the predicted injection functions applying the analytical inverse and iterative stripping methods across (a) uniphasic and (b) biphasic injection protocols. Each block includes 16 different combinations of four ICs (y axis) and four injection durations (x axis).

FIG. 24 is a graph showing an example required injection function to achieve a desired enhancement in the kidneys across a library of 58 XCAT models. Applying the analytical inverse method on a given contrast enhancement in the kidneys (solid line), different injection functions (dashed lines) were calculated for individual XCAT models.

FIG. 25 illustrates graphs showing distribution of peak value of simulated arterial and hepatic contrast enhancement curves from 58 XCAT models for (a) uni-phasic and (b) bi-phasic injection protocols.

FIG. 26 depicts “tissue contribution” of radiation dose increase due to the presence of iodine in different components of vessels network. The distribution was approximated by probability of the secondary electrons which generated inside the iodine and deposited outside the iodine using a Monte Carlo simulation of a simplified organ model.

FIG. 27 depicts Monte Carlo simulation of the organ dose to the heart, spleen, liver, kidneys, stomach, colon, small intestine, and pancreas as a function of time across the XCAT models undergoing a contrast-enhanced abdomen CT scan. Each curve is representing an XCAT model. The organ doses are normalized by CTDIᵥₒₗ.

FIG. 28 shows calculated effective dose as function of time across XCAT models undergoing an abdomen contrast enhanced CT scan. The effective doses are normalized by DLP.

FIG. 29 shows distribution of the maximum dose increment (in percentage) in the heart, spleen, liver, kidney, stomach, colon, small intestine, and pancreas due to the administration of contrast medium across the XCAT models.

FIG. 30 shows potential range of radiation dose alteration for a) liver and b) kidney. The impact of iodine on radiation dose increase varies as the iodinated blood circulates through the vascular system. A normal distribution was assumed for the impact of iodine on dose increase as the blood enters the arteries and flows through the capillaries in which it experiences the maximum proximity to the tissue cells and washes out subsequently via the veins.

### DETAILED DESCRIPTION

The following detailed description illustrates embodiments of the present disclosure and manners by which they can be implemented.

The example functions and methods disclosed herein may be applied to any suitable imaging system, such as a CT imaging system or a magnetic resonance imaging (MRI) system. In the examples described herein, a CT imaging system is referenced but it should be understood that the present subject matter may be applied to any other suitable imaging system that uses contrast material for imaging a target portion of a subject.

A CT imaging system may use a three-dimensional (3D) rotational x-ray examination device having one or more x-ray tubs and the same number of detectors placed on a gantry that is rotated around a patient. Other types of CT imaging systems may use 3D examination devices equipped with a stationary detector ring with multiple pixels that is mounted on the stator of the gantry, and only the x-ray tube is rotating with the rotor of the gantry around the patient. The CT scanning may be based on a rectilinear propagation and attenuation of x-rays. The CT imaging system may thereby acquire a series of x-ray projections from a range of angles around the subject. Each projection can represent the value (or collection of values in a multi-element x-ray detector) of the x-ray attenuation line integral through the object along the line from an x-ray source to an x-ray detector. Imaging an object to be graphically reconstructed at equiangular-spaced views over 180° forms a complete set of projection data. Tomographic image reconstruction can create a two-dimensional (2D) image (or 3D volume) from the measured projection data.

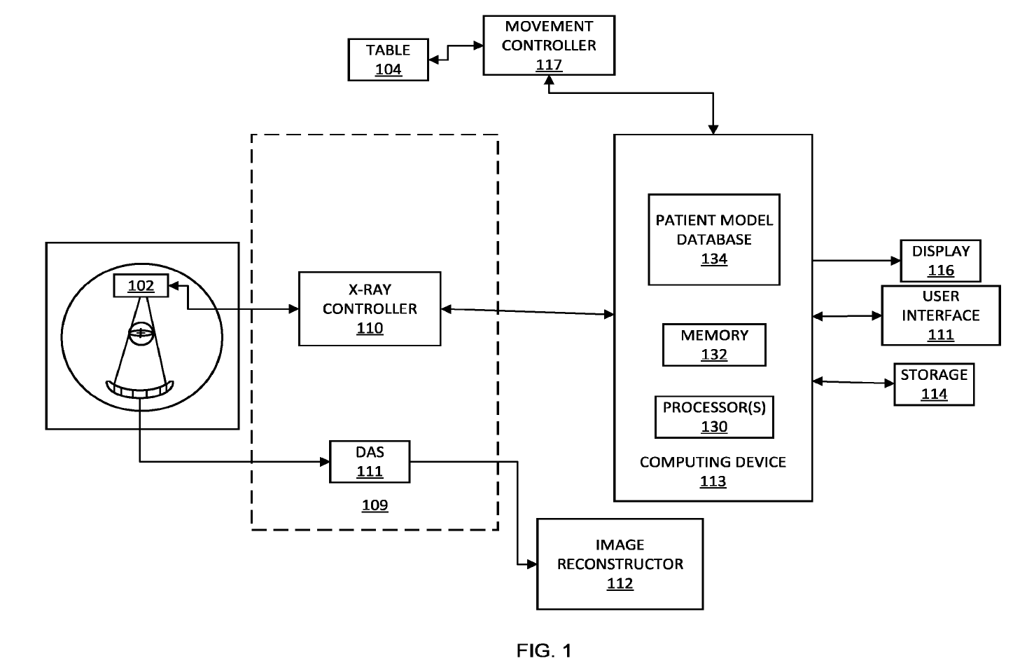

FIG. 1 illustrates a schematic diagram of an example CT imaging system 100 for estimating irradiation dose for patient application based on a radiation field model and a patient anatomy model in accordance with embodiments of the present disclosure. Referring to FIG. 1, the system 100 includes a rotational gantry 101 that is rotatable about a longitudinal axis 108 of a patient's body 107 or any other object to be examined. The gantry 101 may include one or more x-ray sources or tubes 102 that are configured to project a beam of x-rays 106 towards an x-ray detector array 103 placed at the opposite side of the gantry 101. The x-ray detector array 103 can be equipped with multiple detector elements 103a which can together sense the projected x-rays passing through the patient's body 107 to be examined between x-ray detector array 103 and x-ray source 102. Each detector element 103a can generate an electrical signal that represents the intensity of an impinging x-ray beam and can hence be used to estimate the attenuation of the beam as it passes through the object.

In a rotational CT scanner such as depicted in FIG. 1, a 3D volume can be calculated by reconstructing and stacking individual 2D slices. Some CT imaging systems can employ 2D detector arrays, allowing the acquisition of a truly 3D data sets. As shown, only a single row of detector elements 103a is shown (i.e., a detector row). However, a multi-slice detector array such as denoted by reference number 103 include multiple parallel rows of detector elements 103a such that projection data corresponding to multiple quasi-parallel or parallel slices can be acquired simultaneously during a scan. The detector elements 103a may completely encircle the patient 107. This figure shows only a single x-ray source 102, but it should be understood that multiple x-ray sources may be positioned around gantry 101.

Operation of x-ray source 102 can be governed by a control mechanism 109 of the system 100. Control mechanism 109 can include an x-ray controller 110 that provides power and timing signals to one or more x-ray sources 102. A data acquisition system (DAX) 111 belonging to the control mechanism 109 can sample analog data from detector elements 103a and can convert the data to digital signals for subsequent processing. An image reconstructor 112 can receive sampled and digitized x-ray data from DAS 111 and can perform high-speed image reconstruction. The reconstructed image can be applied as an input to a computing device 113 (e.g., a desktop or laptop computer), which stores the image in a mass storage device 114. The computing device 113 may include hardware, software, firmware, or combinations thereof for implementing the functionality described herein. For example, the computing device 113 may include one or more processors 130 and memory 132. The image reconstructor 112 may be specialized hardware residing in the computing device 113 or a software program executed by the computing device 113.

The computing device 113 may receive signals via a user interface or graphical user interface (GUI). Particularly, the computing device 113 may receive commands and scanning parameters from a user interface 115 that includes, for example, a keyboard and mouse (not shown). An associated display 116 can allow an operator to observe the reconstructed image and other data from the computing device 113. The operator-supplied commands and parameters can be used by the computing device 113 to provide control signals and information to the x-ray controller 110, DAS 111, and a table motor controller 117 in communication with a patient table 104, which controls a motorized patient table 104 so as to position patient 107 in gantry 101. Particularly, patient table 104 can move the patient 107 through a gantry opening.

The computing device 113 may be used for implementing functionality described herein. Particularly, for example, the computing device 113 may include hardware, software, firmware, and combinations thereof for implementing the methods and techniques disclosed herein. For example, the methods and techniques may be implemented by the processor(s) 130 and memory 132 as will be understood by those of skill in the art. Further, a user may suitably interact with the user interface 115 for implementing the functions and for presenting results to the user.

In accordance with embodiments of the present disclosure, contrast material can be orchestrated with the scan parameters (e.g. scan timing) based on a relationship between contrast enhancement and injection function. Knowing the dynamics of contrast material distribution, systems and methods are provided that can model injection protocols based on desired contrast enhancements. Such systems and methods can allow for an integrated optimization of contrast medium dose, radiation dose, image quality, and patient-specific contrast administration protocols together. In accordance with the present disclosure, a strategy is provided to determine the required contrast material injection function to achieve a desired contrast enhancement in aorta, kidney, stomach, small intestine, liver, and other anatomical structures by incorporation of a physiologically based compartmental model of dynamics of contrast material distribution.

In accordance with other embodiments, systems and methods are provided that can incorporate the dynamics of blood circulation and thus the perfusion of contrast medium into a library of XCAT models to build towards the next generation of computational patient phantoms, 5D XCAT models with the fifth dimension representing contrast dynamics, enabling virtual studies to evaluate new CT imaging technologies and techniques that involve contrast material. Further, the present disclosure provides an application as a convenient calculator for the estimation of contrast enhancement curves in different organs for a variety of injection and patient parameters.

Contrast enhancement is a key component of CT imaging and offer opportunities for optimization. The design and optimization of new techniques however requires orchestration with the scan parameters and further a methodology to relate contrast enhancement and injection function. Herein, such a methodology is used to develop a method, analytical inverse method, to predict the required injection function to achieve a desired contrast enhancement in a given organ by incorporation of a physiologically based compartmental model. In a study, a method was evaluated across 32 different target contrast enhancement functions for aorta, kidney, stomach, small intestine, and liver. The results exhibited that the analytical inverse method offers accurate performance with error in the range of 10% deviation between the predicted and desired organ enhancement curves. The findings of this study can be useful in optimizing contrast medium injection function as well as the scan timing to provide more consistency in the way that the contrast enhanced CT examinations are performed. As discussed in more detail herein, disclosed techniques can predict the contrast material injection function for a desired organ enhancement curve.

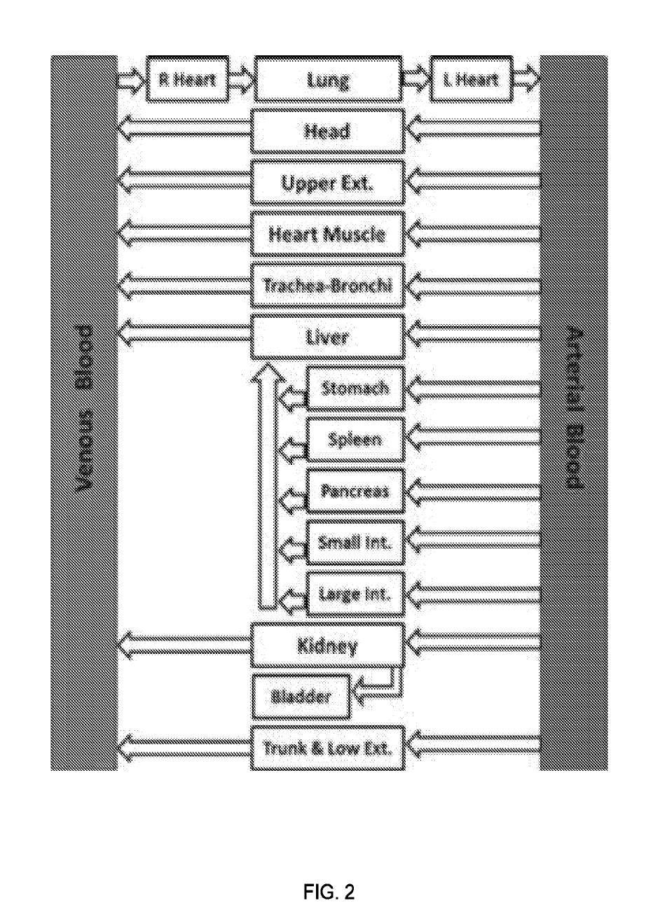

To describe the dynamics of contrast material within the human body, a computer-based physiologic model of the cardiovascular system can be used. FIG. 2 illustrates a diagram of a physiologically based pharmacokinetic (PBPK) compartmental model for simulating distribution pharmacokinetics of contrast medium or material via the cardiovascular system through the body. This model includes 45 compartments, and each arrow represents a vessel compartment and each box represents an organ. The model includes a network of compartmentalized heart, vessels, and organs. This compartmental model (forward model) integrates multiple regulatory factors that govern contrast perfusion such as weight, height, age, gender, cardiac output, and blood volume by incorporating computational pharmacokinetics and known available physiologic data (see Table I). The body flow rate at each compartment, intra vascular, and extra cellular volumes, based on experimental measurement from a normal male patient with weight of 68 (kg) and height of 170 (cm) are provided. The enhanced blood is assumed to be a uniform mixture of blood and contrast material. As this mixture reaches a compartment, it is distributed through the compartment and subsequently washed out. Within this process, the flow rate and concentration of the contrast material within blood is changing. A mass balance described by Fick's law of diffusion is applied to each compartment as a part of an integrated system. The concentration of contrast material in each organ was calculated by solving a series of differential equations defining the forward model. As disclosed herein, a technique can be used to predict the injection function to provide a targeted enhancement curve of an organ or vessel.

TABLE I
Compartmental model parameters. Type of compartment (i.e., vessel or organ),
body flow rate at each compartment, intra vascular and extra cellular volumes based on
experimental measurement from a male patient with weight of 68 (kg) and height of 170
(cm) were extracted from literature.

| | | BFR* | IVV** | ECV** | | | BFR | IVV | ECV |
|---|---|---|---|---|---|---|---|---|---|
| Compartment | Type | (ml/sec) | (ml) | (ml) | Compartment | Type | (ml/sec) | (ml) | (ml) |
| 1. Inject Vessel | vessel | 2.7 | 40 | 0 | 20. Vessel 20 | vessel | 15.5 | 20 | 0 |
| 2. Right heart | vessel | 29.0 | 180 | 0 | 21. S. Intestine | organ | 15.6 | 20 | 322 |
| 3. Vessel 3 | vessel | 108.3 | 130 | 0 | 22. L. Intestine | organ | 15.6 | 14 | 218 |
| 4. Lung P | organ | 108.3 | 150 | 144 | 23. Vessel 23 | vessel | 8.2 | 20 | 0 |
| 5. Vessel S | vessel | 108.3 | 160 | 0 | 24. Stomach | organ | 8.25 | 10 | 62 |
| 6. Left Heart | vessel | 108.3 | 180 | 0 | 25. Vessel 25 | vessel | 23.5 | 100 | 0 |
| 7. Aorta | vessel | 108.3 | 100 | 0 | 26. Liver | organ | 7.5 | 71 | 524 |
| 8. Vessel 8 | vessel | 16.3 | 20 | 0 | 27. Vessel 27 | vessel | 31.4 | 100 | 0 |
| 9. Head | organ | 16.3 | 37 | 484 | 28. Vessel 28 | vessel | 80.1 | 800 | 0 |
| 10. Vessel 10 | vessel | 16.3 | 80 | 0 | 29. Pancreas | organ | 8.25 | 2 | 12 |
| 11. Vessel 11 | vessel | 5.4 | 20 | 0 | 30. Spleen | organ | 8.25 | 6 | 37 |
| 12. Up. Extremity | organ | 5.4 | 12 | 2751 | 31. Vessel 31 | vessel | 48.7 | 80 | 0 |
| 13. Vessel 13 | vessel | 2.7 | 40 | 0 | 32. Vessel 32 | vessel | 23.8 | 20 | 0 |
| 14. Vessel 14 | vessel | 2.7 | 40 | 0 | 33. Kidney | organ | 23.5 | 54 | 89 |
| 15. Heart Muscle | organ | 4.3 | 10 | 103 | 34. Vessel 34 | vessel | 23.8 | 100 | 0 |
| 16. Vessel 16 | vessel | 82.3 | 100 | 0 | 35. Vessel 35 | vessel | 48.7 | 700 | 0 |
| 17. Lung NP | organ | 2.2 | 5 | 144 | 36. Vessel 36 | vessel | 24.9 | 200 | 0 |
| 18. Vessel 18 | vessel | 80.2 | 100 | 0 | 37. Trunk/Low Ext | organ | 24.9 | 57 | 11002 |
| 19. Vessel 19 | vessel | 7.5 | 20 | 0 | 38. Vessel 38 | vessel | 24.9 | 1000 | 0 |

### Analytical Inverse Method

The inverse method is interpreted as Fick's law description of iodine concentration balance applied as

V(dCₒᵤₜ/dt)=Q(Cᵢₙ−Cₒᵤₜ) (1)

for a vessel compartment and as

V_{IV}(dC_{IV}/dt)=Q(Cᵢₙ−C_{IV})−PS(Cᵢₙ−C_{EC}), (2.a)

V_{EC}(dC_{EC}/dt)=PS(C_{IV}−C_{EC}), (2.b)

and Cₒᵤₜ=(M_{IV}+M_{EC})/Vₜₒₜ (2.c)

for an organ compartment. In these equations, V, Q, Cᵢₙ, Cₒᵤₜ, M, P, S, and t are volume, blood flow rate, input concentration, output concentrations, mass of contrast medium, vascular membrane permeability, surface area, and time, respectively. Knowing the concentration function of iodine in the compartment, this method can allow for prediction of the injection function as the input concentration in the intra vascular injected vessel by solving the differential equations for the compartments in the path of interest (i.e., the set of compartments which included the target organ or vessel) successively from the target compartment back towards the first compartment (injected vessel) along the path. Finally, the injection function may be created by converting the estimated continuous function into a rectangular function. Using the same plateau of the estimated continuous function, the width of the rectangular function may be calculated based on the area under the curve.

For evaluation purpose, 32 different injection protocols as the combination of 4 different iodine concentrations, 4 different injection duration, and 2 injection bolus shapes (uni-phasic and bi-phasic) were created. The uni-phasic and bi-phasic injection protocols used for the evaluation are summarized in Table II. Target desired organ enhancement (OE_{desire}) in aorta, kidney, stomach, small intestine, and liver were determined as the targeted enhancement functions. Using the analytical inverse method, the injection functions were predicted (IF_{predict}) from the targeted organ enhancement functions. Applying the forward compartmental model on IF_{predict}, predict, the predicted organ enhancement (OE_{predict}) curves were then calculated. The accuracy of the predicted organ enhancement results were evaluated and compared across different protocols.

TABLE II
Contrast material administration protocols. 32 different injection
protocols as the combination of 4 different iodine concentrations, 4
different injection duration, and 2 injection bolus shapes (uni-phasic
and bi-phasic) were studied.

| | Iodine | | Injection | Injection |
|---|---|---|---|---|
| | Concentration | Volume | Duration (First | Duration |
| Protocol | (mgI/ml) | (ml) | Phase) | (Second Phase) |
| Uni-phasic | 300, 320, 350, 400 | 125 | 20, 25, 30, 35 | 0 |
| injection | | | | |
| Bi-phasic | 300, 320, 350, 400 | 125 | 20, 25, 30, 35 | 75, 70, 65, 60 |
| injection | | | | |

For comparison between the desired and predicted organ enhancement curves, three different error metrics were used: (1) Time-to-peak error (%), (2) peak-value-error (%), and (3) mean absolute error (MAE in %). Mean absolute error was defined as

$$MAE = \frac{{\sum }_{t = 1}^{n} \vert {OE}_{desire , t} - {OE}_{predict , t} \vert}{{OE}_{desire , t}} \cdot 100 \% \qquad ( 3 )$$

Error maps were created for kidney by calculating and using the errors across the entire uni-phasic and bi-phasic protocols. Each error map block included 16 injection protocols, i.e. each block is consisted of 16 different combinations of 4 iodine concentrations and 4 injection durations. Furthermore, using the maximum value of different error metrics, the error bars were created across all the organs and injection protocols studied.

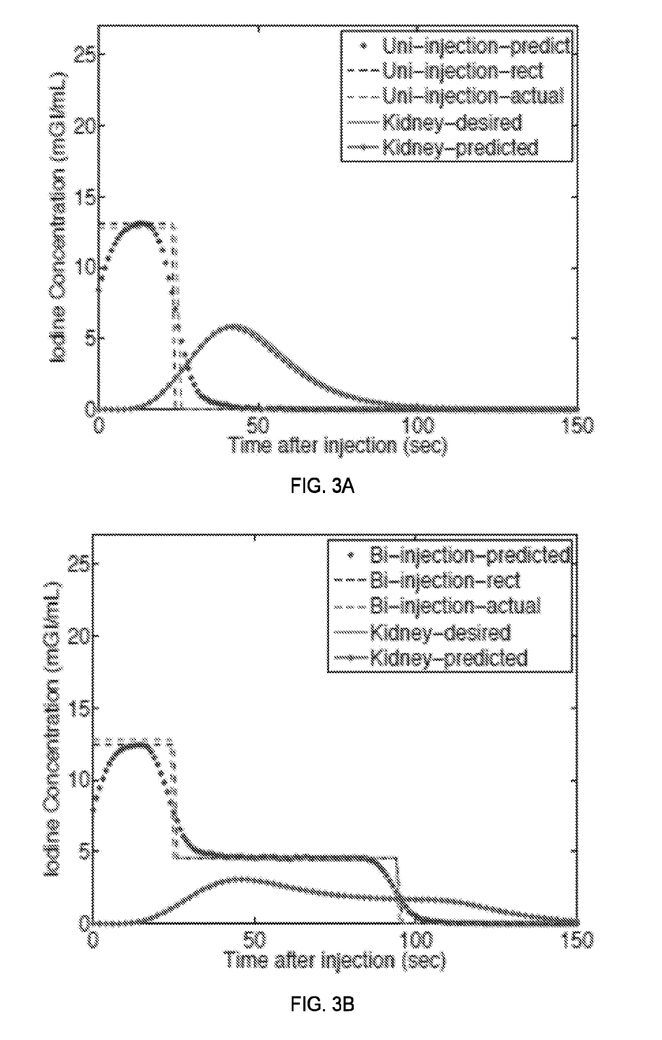

Applying the analytical inverse method, desired injection functions as well as predicted and desired organ enhancement curves for aorta, kidney, liver, stomach, and small intestine were calculated based on inputted 32 original injection functions. FIGS. 3A and 3B show the enhancement curves calculated in kidney on one selected uni-phasic (injection of 125 mL of contrast medium of 320 mgI/ml iodine concentration at 5 mL/s) as well as one selected bi-phasic (injection of 50 mL of the same contrast medium at 2.5 mL/sec followed by 75 mL at 1 mL/sec) injection protocol.

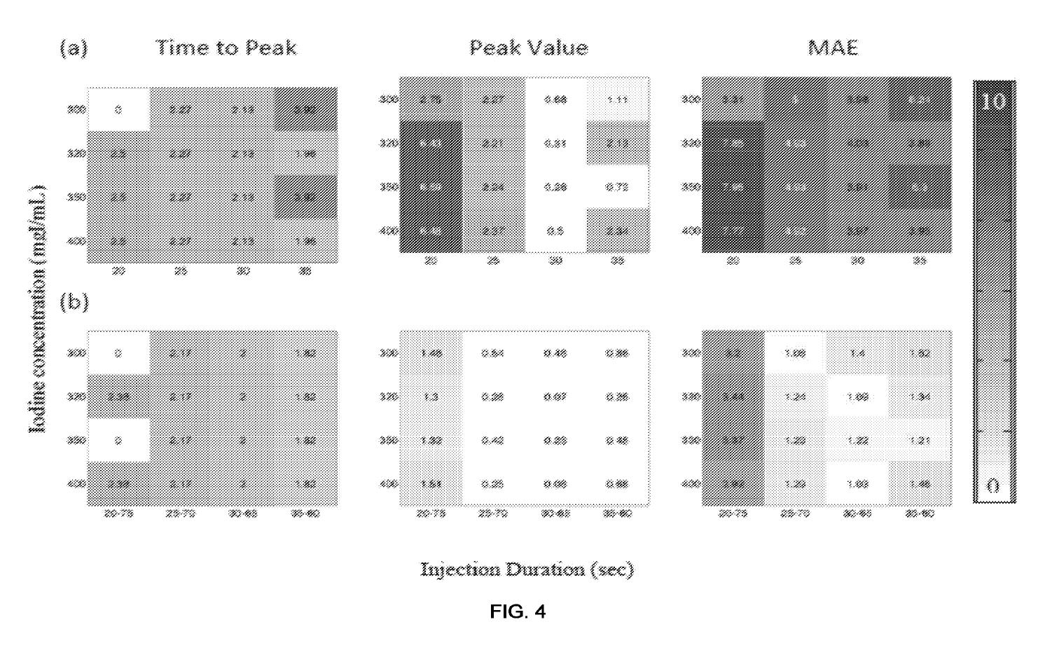

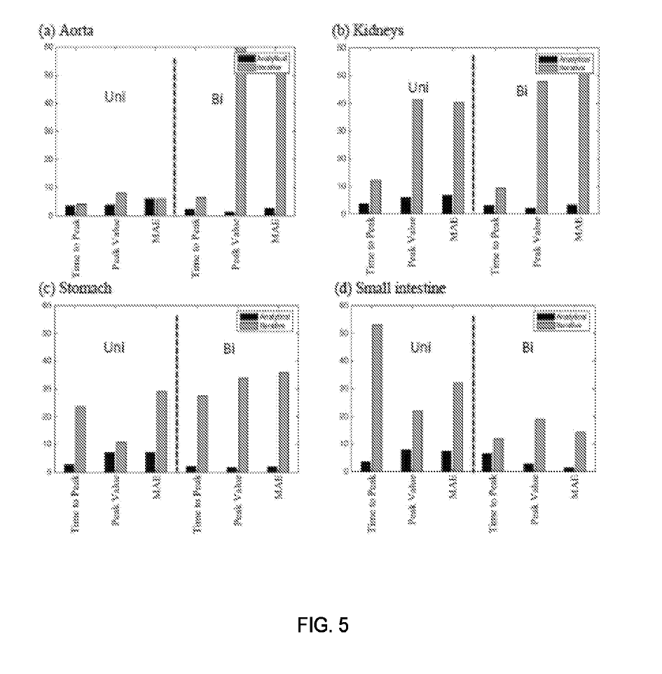

FIG. 4 shows the error map generated using the time-to-peak, peak value, and mean absolute errors calculated across uni-phasic (FIG. 3.a) and bi-phasic (FIG. 3.b) injections. FIG. 5 compares the maximum value of each error metrics for all the organs across all 32 injection functions. The maximum value of time-to-peak, peak-value, and mean absolute error applying the analytical inverse method across uni-phasic protocols were 3.3%, 4.0%, 6.8% for aorta, 3.9%, 6.9%, 7.9% for kidney, 3.4%, 7.2%, 7.9% for stomach, and 3.1%, 7.7%, 8.0% for small intestine. For bi-phasic injection protocols, the errors were 3.2%, 0.8%, 2.0% for aorta, 2.6%, 1.5%, 3.44% for kidney, 1.8%, 1.3%, 2.4% for stomach, and 7.2%, 2.0%, 2.4% for small intestine. Analytical inverse method, per design, was incapable of predicting the injection function based on the liver enhancement.

Many clinical practices utilize intravenous contrast medium enhancement techniques for body CT to improve the image quality. To reach the consistency in contrast enhanced image acquisition, the concordance of the organ enhancement time and time of the scan can be extremely important. However, the current practice of contrast medium administration is variable, often not reflective of the patient's attributes and thus making quantitative analysis of the contrast medium enhancement difficult. Herein, applying the knowledge of mechanism of contrast enhancement, methods, analytical inverse methods, are provided to determine the required injection function to achieve the targeted contrast enhancement in a specific organ.

In experimentation, the predicted organ enhancement results from analytical inverse technique showed encouraging agreement with desired organ enhancement results. The maximum value of time-to-peak, peak value, and mean absolute errors across the studied organs was less than 8.0% for uni-phasic injection protocols. In general results from bi-phasic protocols were more accurate, which can be explained as the result of the longer injection duration of the bi-phasic (95 seconds) in contrast to the uni-phasic injection duration (25-35 seconds), and hence more data points. Considering the accuracy level of results across different organs (<8%), analytical inverse model can be considered as robust method to predict the right injection function to obtain the targeted contrast enhancement.

The error map generated for different organs from an analytical inverse method share a characteristic. Generally speaking, the variability in injection time results in more discrepancies in errors than variability in iodine concentration, i.e., more change in color along the individual rows of each error metric block for a specific organ in FIG. 4 than along the individual columns. This can be explained by the same nature of the iodine concentration of the injected contrast medium and the calculated iodine concentration, i.e., the contrast enhancement in the organ, while the relationship of the duration of injection with contrast enhancement is more complicated.

Disclosed herein are analytical inverse methods for determining the required contrast medium injection function to achieve a desired contrast enhancement in specific organs by incorporation of a physiologically based compartmental model of dynamics of contrast material distribution. By applying such methods, better than 10% accuracy can be achieved in delivering an organ enhancement based on predicted injection function. Such an approach can be useful in optimizing contrast medium injection function as well as the scan timing to provide more consistency in the way the contrast enhanced CT examinations are performed.

In accordance with embodiments, systems and methods are disclosed that provide, in part, a PBPK model of the human cardiovascular system that was incorporated into a library of 58 extended cardiactorso (XCAT) patient models framework. To do so, a physiological compartmental model of the blood circulation network was developed in which each compartment represents a vessel or organ. To construct the PBPK model for various patient anatomies, known XCAT organ volumes were extracted from each model and incorporated into models. The total blood volume was predicted for individual XCAT models by regression formulas. For a given injection protocol and XCAT model, the contrast medium dynamics within the body was described by a series of mass balance differential equations, the solutions to which provided the contrast material concentration time curves for each organ. Finally, for each patient, a library of time dependent organ materials was defined. Using this library, each organ was assigned to a corresponding time varying material to create the first generation of contrast material enhanced computational phantoms, 5D XCAT phantoms, in which the fifth dimension represents the dynamics of contrast material.

The presently disclosed subject matter involves the integration of a physiologically-based compartmental contrast model into a computational human phantom framework to simulate contrast dynamics for CT imaging studies. Described herein is a contrast model and how it was tailored to specific patient anatomies for various XCAT phantoms.

The dynamics of contrast material can be simulated via PBPK models of the cardiovascular system. The cardiovascular system regulates the distribution of contrast material within the blood throughout the body after injection. PBPK modeling enables us to relate the concentration of compound substances (e.g. drug, contrast agent, etc.) within internal tissues to the administered dose of chemical compounds. This relationship, however, is intrinsically complicated by two interacting factors: the patient (i.e. height, weight, cardiac output, tissues volumes, age, gender), and contrast medium injection technique (concentration, volume, injection rate, injection bolus and saline chaser).

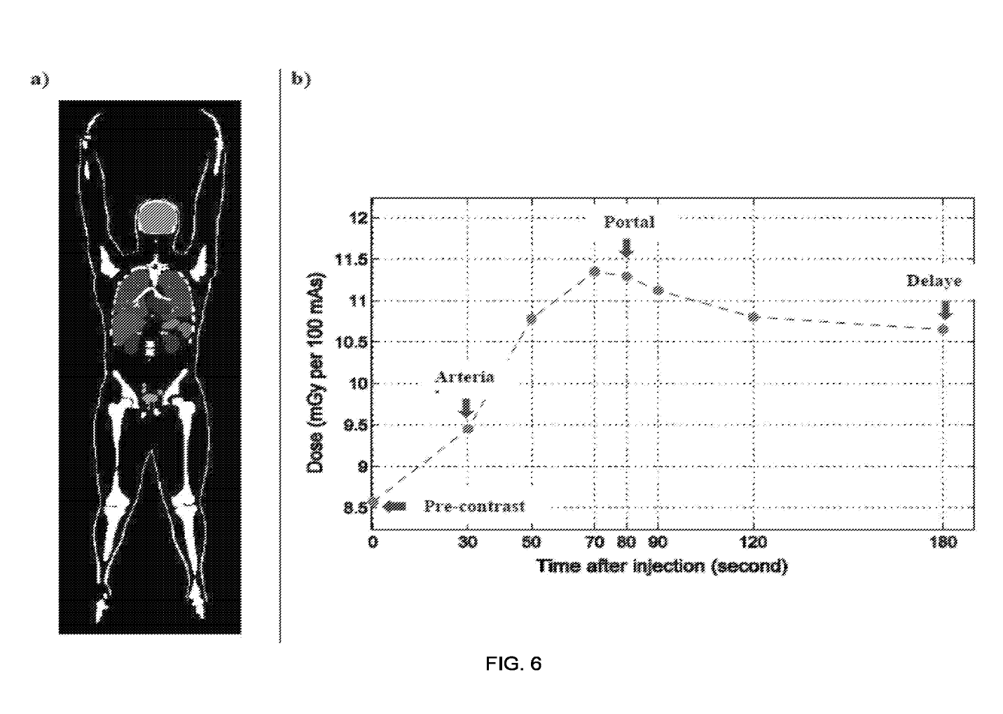

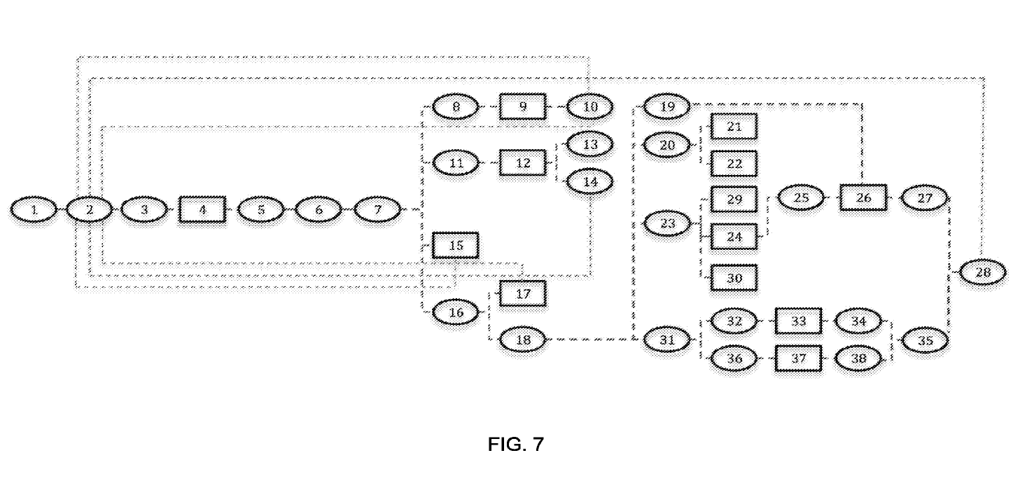

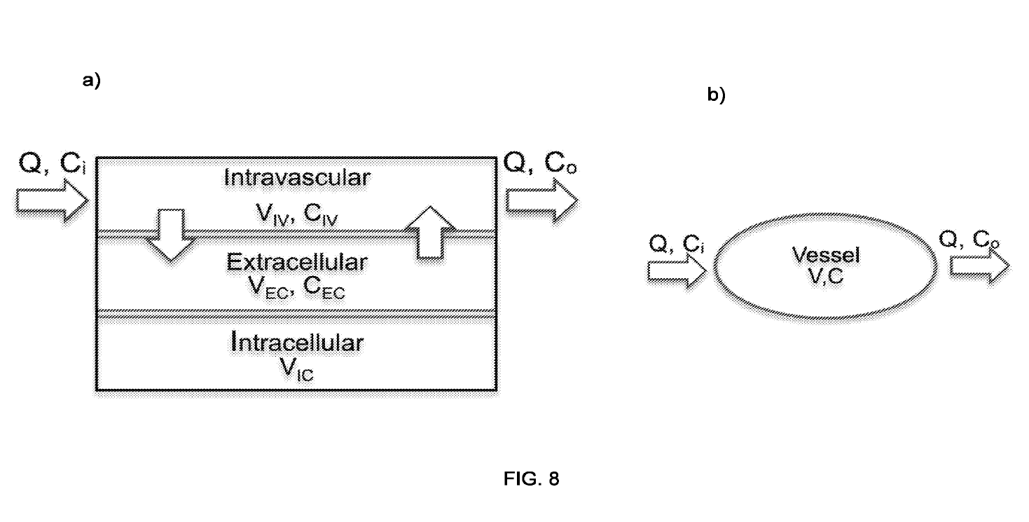

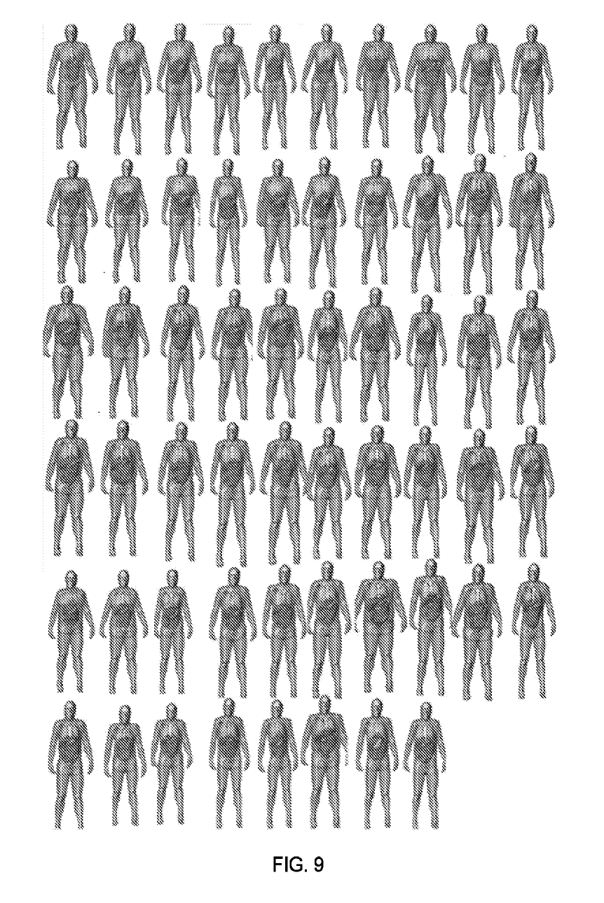

In accordance with embodiments, we adopted a PBPK technique is used for the prediction of vascular and parenchymal contrast enhancement rates. The technique may be based on sets of equations that model the time courses of chemicals transported to and from various tissues via the blood throughout the body. The model consisted of a network of compartments representing the key organs and vessels as shown in FIG. 6. The parameters of this model were adjusted to personalize it to fit various patient anatomies of the XCAT models. Referring to FIG. 6, the figure shows the PBPK compartmental model used for simulating pharmacokinetics of contrast medium via the cardiovascular system through the body. The model includes 38 compartments: each ellipse represents a vessel compartment and each box represents an organ (see Table I).

To construct the PBPK model for each XCAT phantom, a set of initial adjustments was required. This primary model with initial estimated and collected values is referred to as the standard contrast model. The average blood volume of 5 liters (3 L of plasma and 2 L of red blood cells) and cardiac output of 6 liters per minute were used in this standard model. Each individual's tissue volume, blood flow rate, and blood distribution at different regions as well as the distribution of body fluid including intravascular (IV), extracellular (EC), and intracellular (IC) were determined from available physiological data. For a 70-kg adult patient, the total body fluid was assumed to be 40 L. Depending on the contrast material injected, the EC fluid volume ranges from 9 to 22 L. The EC fluid volume decreases as the molecular weight of the contrast material increases. The total EC fluid volume (including 3 L of plasma) used in this model was 18.9 L (calculated using 0.27 as the apparent volume value of distribution of iohexol), i.e., 15.9 L without plasma. For a typical adult patient with blood volume of approximately 5 liters, the total IC fluid volume (19.1 L) was calculated as the difference of the total body fluid (40 L) and total EF fluid volume (18.9). In the absence of information about EC and IC fluid volume for individual organs, an assumption was made to estimate the fluid volumes of individual organs using the total fixed ratio of 0.83, which is the ratio of the total EC fluid volume and IC fluid volume, i.e. 15.9/19.1=0.83. Finally, the capillary volumes and regional blood flow rates were assigned based on available physiologic data. The estimated blood distribution in the vascular system, blood flow rate, and capillary volumes are listed in Table I.

As a first order approximation, contrast-enhanced blood was assumed to be a uniform mixture of blood and contrast material propagating through a web of blood vessels and organ compartments. As this mixture reaches the compartment, it is evenly distributed through the compartment and subsequently washed out. Within this process, the concentration of the contrast material in the blood is changing. In order to evaluate the change in concentrations of contrast material in blood, each compartment, organ or vessel, was assigned to a corresponding mass balance differential equation. The generic mass balance equation used for a vessel compartment was

$$\frac{dA}{dt} = ( {Q}_{i} \cdot {C}_{i} ) - ( {Q}_{o} \cdot {C}_{o} ) , \qquad ( 1 )$$

where A is the mass of contrast material in the tissue, t is the time, Cᵢ and Cₒ are the input and output concentrations, and Qᵢ and Qₒ are the input and output flow rates. In this model, a blood vessel is considered as a well-stirred compartment with a constant volume

$$( \frac{dV}{dt} = 0 )$$

and single inlet and outlet flow rates, and hence Qᵢ=Qₒ=Q. So Equation (1) can be reformulated into a first-degree differential equation a

$$\frac{{dC}_{o}}{dt} = \frac{Q}{V} ( {C}_{i} - {C}_{o} ) , \qquad ( 2 )$$

which simply states that the rate of change in the amount of contrast material with respect to time is proportional to the difference between the rates by which the material enters and leaves the compartment.

As noted earlier, each organ compartment was assumed as three interacting sub-compartments: intravascular, extracellular, and intracellular. Each sub-compartment was modeled as a single well-mixed compartment. As a result, contrast material mass transfer occurs due to not only the transport of blood, but also the diffusion through the membrane of extra- and intracellular spaces. For a membrane, the mass transfer rate was formulated by

$$\frac{dM}{dt} = PS * ( {C}_{i} - {C}_{o} ) , \qquad ( 3 )$$

where M is iodine mass and PS is the product of permeability degree and surface area of the membrane. In this work, since iodinated contrast material usually does not diffuse through the cells, diffusion was only considered for intravascular and extracellular spaces. Therefore, for an organ compartment, a set of two mass balance equations were employed

$$\begin{matrix}\frac{{dC}_{IV}}{dt} = \frac{1}{{V}_{IV}} [ Q ( {C}_{i} - {C}_{IV} ) ] - \frac{1}{{V}_{IV}} [ PS ( {C}_{IV} - {C}_{EC} ) ] , \\ and & ( 4. a ) \\ \frac{{dC}_{EC}}{dt} = \frac{1}{{V}_{EC}} [ PS ( {C}_{IV} - {C}_{EC} ) ] , & ( 4. b )\end{matrix}$$

where Q, Cᵢ, t, and P are described above, C_{IV} and C_{EC} are intravascular and extracellular concentrations, and V_{IV} and V_{EC} are intravascular and extracellular volumes. The first equation was assigned to the intravascular sub-compartment and the second equation to the extracellular sub-compartment. Note that the first term on the right side of the Equation (3.a) describes the blood-flow mass balance, as it is proportional to difference of the input concentration and intravascular concentration, while the second term describes the diffusion of contrast material through the membrane of the capillaries. Finally, for the extracellular space, there is only one term describing the contrast diffusion within the extracellular space.

With specific parameters and equations assigned to the compartments, the compartments were integrated into a single computational system. Contrast material is typically injected through the antecubital vein; it then mixes with the blood and flows into the right side of the heart and finally distributes throughout the body. In an example model, this distribution was described by a series of differential equations, the solutions to which can provide the iodine mass delivery rate to each tissue. The iodine mass in each compartment represents the accumulation of injected contrast material in addition to the recirculated contrast medium in the cardiovascular system. Two rounds of circulation were modeled in this study. The coding was done through a scientific computing platform (MATLAB R2014a, Mathworks). The equations were solved using Runge-Kutta method built-in MATLAB function, ode45.

As the next step in this method, the concentration of iodine in each organ was calculated from the ratio of the total mass of contrast material within the organ to the organ volume. The total mass of contrast medium in an organ was determined from the summation of the solutions of the Equation (3.a) and (3.b), C_{IV} and C_{EC}, multiplied by V_{IV} and V_{EC}, respectively. Finally, intravascular, extracellular, and intracellular volumes were added to calculate organ volume, and hence the iodine concentration.

In order to create a population of 5D XCAT phantoms, including contrast dynamics, patient-specific PBPK contrast models were implemented into previously developed XCAT models. For one study, use of 58 adult XCAT patient models from a library (FIG. 9) with a mean age of 52 years (age range, 18-78), mean weight of 80.2 kg (weight range, 52-117); including 35 men (age range, 18-78 years; weight range, 60-117 kg) and 23 women (age range, 27-75 years; weight range, 52-106 kg). The following steps summarize this method. First, to construct the patient-specific contrast model for each phantom, the known organ volumes were extracted from each specific XCAT model and used to correct the entire regional blood volumes of the organs (including intravascular, extracellular, and intracellular spaces) in the PBPK standard model. As the organs within the XCAT phantoms are largely homogenous, to assess the organ sub-compartments volumes, the fixed ratio of 15.9/19.1 was assumed for the extracellular and intravascular space volumes.

Second, as not all blood vessels are currently modeled in the XCAT phantoms, the total blood volume was predicted for each XCAT model using BVₘₐₗₑ=23.6×H^{0.725}×W^{0.425}−12 and BV_{female}=24.8×H^{0.725}×W^{0.425}−1954, where BV, H, and W are blood volume, height, and weight of patient. For each XCAT model, the ratio of the predicted blood volume to the standard model blood volume was applied to adjust the volume of the blood vessels. The cardiac output of each model was further adjusted based on the height and weight of the patient using CO_{≤30}=25.3×H^{0.725}×W^{0.425} and CO_{>30}=CO_{≤30}×(1−0.008×(age−30)), where CO_{≤30} and CO_{>30} are cardiac outputs of patients younger and older 30 years.

Each XCAT patient model was defined by a pair of voxelized matrices, one of which defines a map of the organs (i.e., xcat.org file), and the other a profile of corresponding organ materials (i.e., xcat.mat file). In the .org files, the organs and structures were assigned unique integer ID's. The .mat files contain the corresponding iodine densities assigned to the different voxels at a given time point. In order to incorporate the dynamics of blood flow and hence the contrast material distribution into the models, we generated a time series of xcat_*.mat files (e.g., xcat_0.mat, xcat_5.mat, xcat_20.mat files, in which the file number represents the time (seconds) after the injection) corresponding to different time points after the injection for each xcat*.org file.

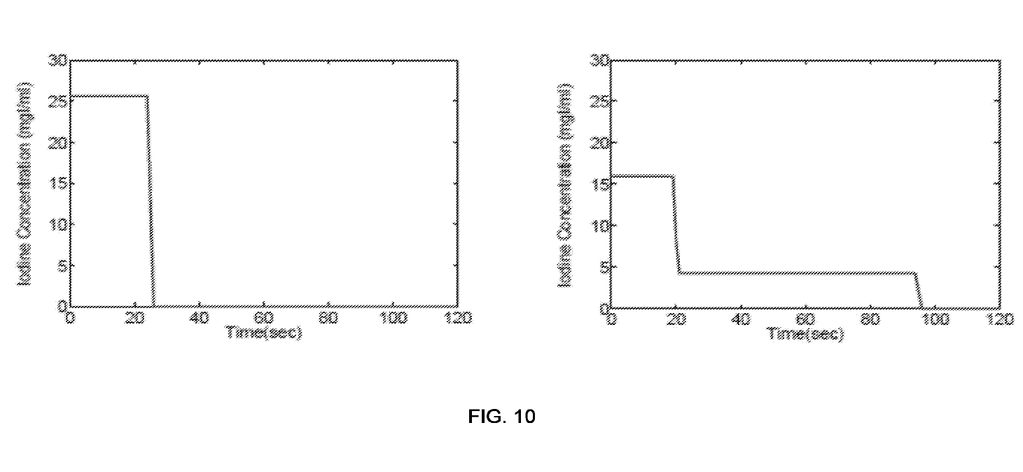

A series of simulations were performed to compare the prediction of the model to clinical data. The intravenous injection protocols used in this work were selected among the most commonly used protocols, a uni-phasic injection of 125 mL of contrast material (320 mgI/mL) at 5 mL/sec and a bi-phasic injection of 50 mL of the same contrast agent at 2.5 mL/sec followed by 75 mL at 1 mL/sec (FIG. 10). The predicted aortic and hepatic results were then compared with clinical data. We compared the simulated results from: (a) an individual 5D XCAT model (70.5 kg weight and 168 cm height), and (b) the library of XCAT models (58 phantoms), subjected to the described injection protocols. The clinical data were obtained from a prior study by Heiken et al. The data reflected the hepatic and aortic mean contrast enhancement values acquired from a group of 27 and 28 patients who underwent the uni-phasic and bi-phasic injection protocols.

Furthermore, the results were compared using four metrics; (1) the mean percentage discrepancy (MP), defined as

$$MP = \frac{1}{n} {\sum }_{i = 1}^{n} \frac{{CE ( i )}_{simulated} - {CE ( i )}_{clinical}}{{CE ( i )}_{clinical}} \cdot 100 , \qquad ( 5 )$$

where n is the number of time points and CE(i) is the contrast enhancement at the ith time point. In addition we used the percent relative discrepancy in (2) the time-to-90% peak values (TP), (3) the peak values (PV), and (4) the slope of enhancement (S). These were calculated for the mean value of simulated and clinical aortic and hepatic contrast enhancements. Furthermore, for each hepatic and aortic pair of simulation and clinical data, a statistical comparison of the simulated and clinical data was performed with a paired t-test at the 95% significance level under the null hypothesis that there was no difference in simulated and clinical data. Such a statistical test is justified because the compared quantities were averaged over different population of patients and assumed to follow Gaussian distribution.

Note that the output of the mass balance differential equations described in Equations 2 and 3 are in the form of contrast material concentration. Therefore, in order to calculate the contrast enhancement, we applied the previously derived proportionality between the iodine concentration and contrast enhancement. For a scanner operating at 120 kVp, the most commonly used tube voltage in adult patients, there is a linear correlation between concentration and contrast enhancement at a constant slope of 25 to 30 HU/mgI. The proportionality constant used in this work was 26.18 HU/mgI.

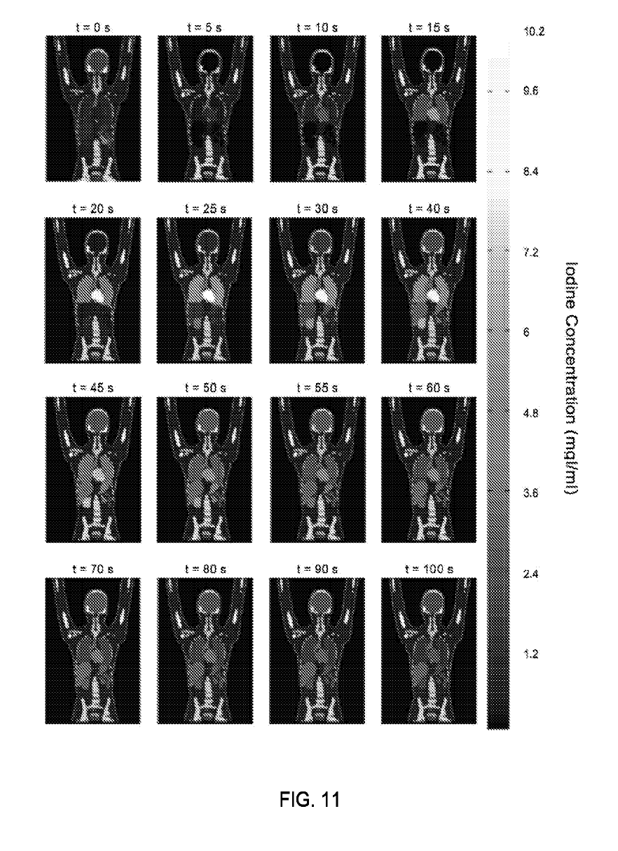

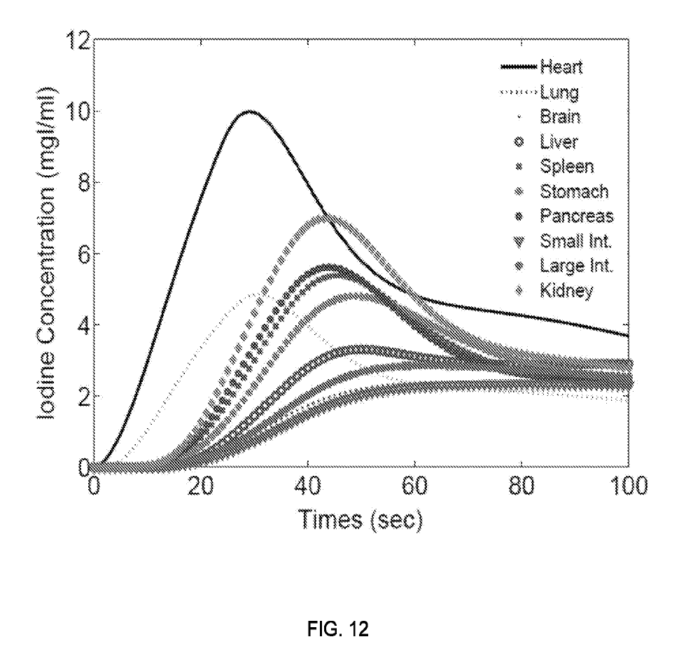

The XCAT series of phantoms were successfully modified to enable simulation of contrast-enhanced CT scans. FIG. 11 shows a collage of a 5D XCAT model at discrete times after administration of contrast material. The organ-voxels value (grayscale bar) in the figure reflect the iodine concentration at different time points from our PBPK model personalized to a selective male XCAT model (with 70.5 kg weight and 168 cm height). The iodine concentration-time results obtained from the brain, heart, lung, liver, stomach, spleen, pancreas, small intestine, large intestine, and kidney are illustrated in FIG. 12.

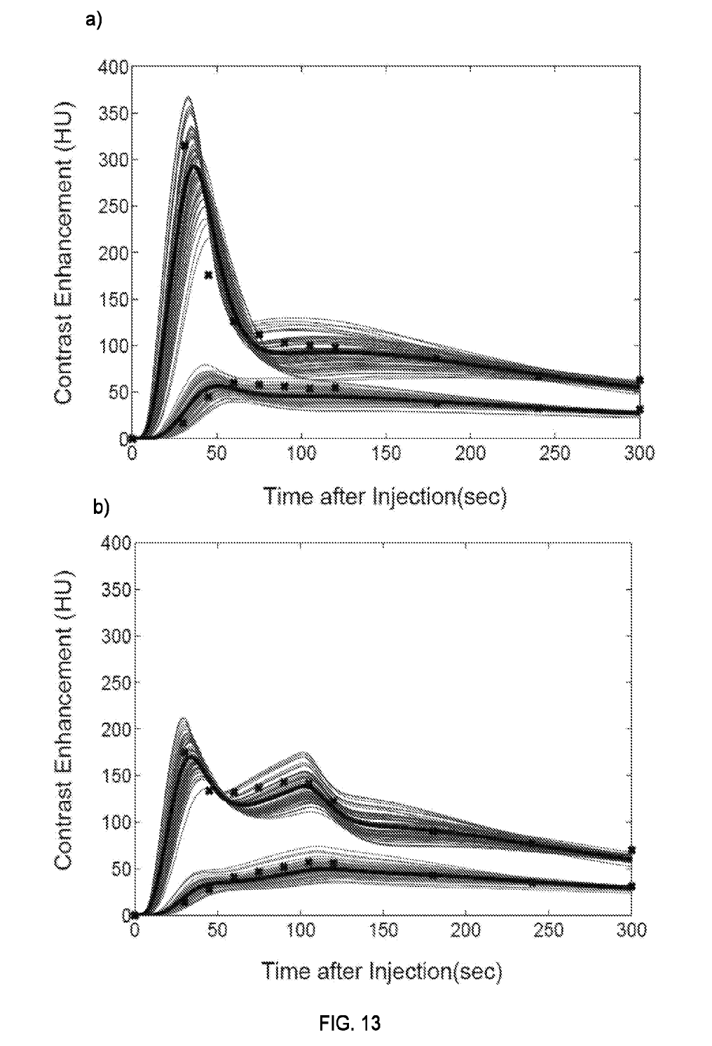

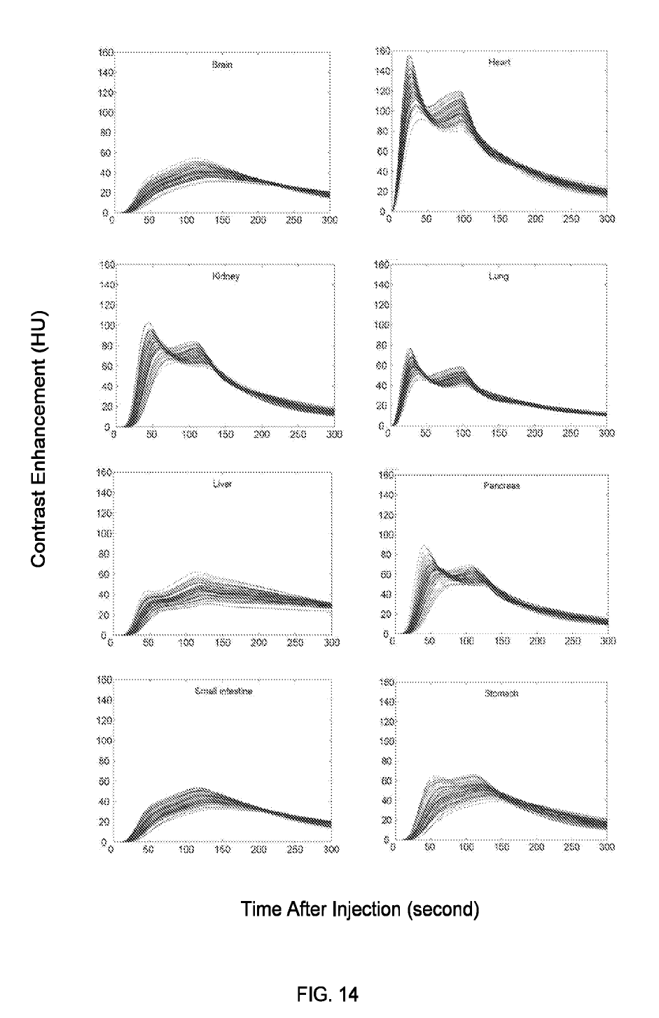

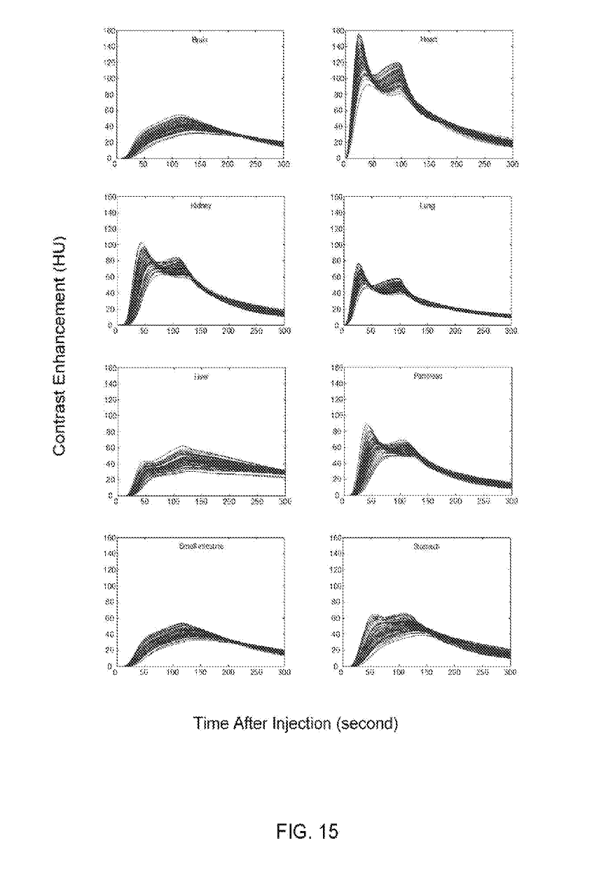

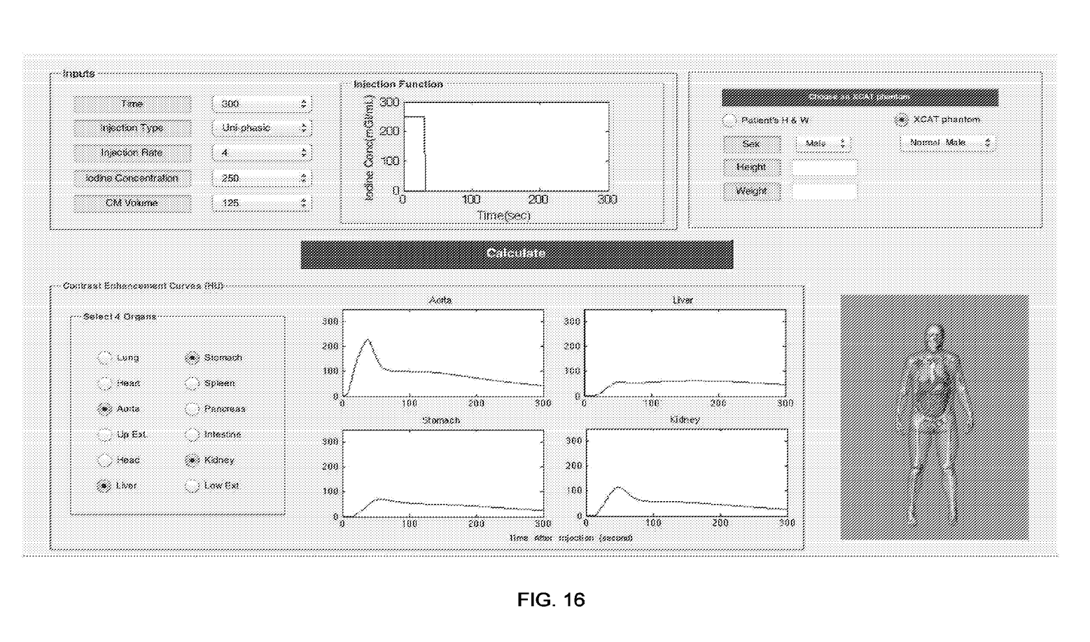

The simulated contrast enhancement-time results from our 5D XCAT models demonstrate encouraging concordance to the clinical data. FIG. 13 shows the simulated aortic and hepatic contrast enhancement-time results from individual XCAT models and also the mean value over all the models compared to the mean clinical contrast enhancement data. The calculated absolute value of MP, TP, PV, and S were 10% (P>0.98), 4%, 7%, and 14% for the aorta and 16% (P>0.74), 25%, 5%, and 18% for the liver, respectively, using a uni-phasic injection. For a bi-phasic injection, the calculated MP, TP, PV, and S were 8% (P>0.66), 1%, 1%, and 12% for the aorta and 12% (P>0.56), 4%, 12%, and 7% for the liver, respectively. The contrast enhancement-time curves for different organs across our library of XCAT phantoms undergoing uni-phasic and bi-phasic injections are shown in FIGS. 14 and 15.

Considering the growing number of CT technologies, the need for virtual clinical trials to optimize image quality versus radiation dose is at an all-time high. VCTs require realistic phantoms to accurately model the human population. Given the wide use of contrast-enhanced imaging, these phantoms also need to be able to model contrast dynamics. With the goal of developing patient-specific computational phantoms with a sufficient level of realism to enable VCT's, next generation of computational patient phantoms, 5D XCAT phantoms, were created that incorporate the dynamics of contrast material distribution within the human body. These new models provide the ability to simulate personalized contrast-enhanced CT imaging procedures with potential applications beyond CT.

The simulated aortic and hepatic contrast enhancement-time values from the enhanced 5D XCAT models were in close agreement with clinical data in terms of the time-to-peak and peak-values. The higher discrepancy values (TP of 25% and S of 18%) calculated for the hepatic enhancement following a uni-phasic injection may be due to the lack of clinical scan data between 45 and 60 seconds, i.e., the missed data points at exact enhancement peak may result in large discrepancy between the simulation and clinical data. The simulated results showed a wide range of dispersion around the mean value (PV range, −22% to 23%) which reflects the inherent anatomical and physiological variability across a population of patients. It is worth mentioning that the difference between the simulation results and clinical data should not be considered as “error”, in that the data compared are from different group of patients.

Despite the fact that the simulated contrast enhancement curves from different organs were different, they all shared a generic pattern, in which the contrast enhancement started from zero followed by a relatively rapid peak with subsequent slow decline. In some organs, the enhancement peak time was affected also by recirculation of the contrast material, which, depending on the injection duration, organ diffusion rate, and location of the organ in the circulatory system with respect to the heart, can increase the enhancement. This was partially evident for the liver. These generic behaviors can serve as a basis for personalizing contrast material injection protocols for individual patients based on their unique attributes.

An application of 5D XCAT models and contrast enhancement estimation technique is a user interface application, XCAT contrast, which can be used to estimate the approximate patient-specific contrast enhancement in different organs for a given CT injection protocol (FIG. 15). The XCAT contrast application was scripted in MATLAB to apply the contrast material injection parameters such as injection type (uni-phase or bi-phase), iodine concentration, volume, and injection rate as the inputs and also the patient factors such as height, weight, and gender to predict the contrast enhancement within the different organs as a function of time after the injection. Patients also can be chosen from the library of XCAT models.

This wide range of variability in cardiac output can have a profound impact on the contrast enhancement results, ranging up to 30%. In work described herein, it is assumed that the cardiac output (range, 3975-7972 ml/min) of the models are related on the height, weight, and age using a regression model. This assumption was made since our study aimed to validate the models against data from a group of patients with no cardiac disease and hence normal cardiac outputs. A single XCAT model cannot be expected to generically represent individual patients with similar characteristics; patients with similar height, weight, and age do have varying cardiac outputs. However, the model can be parameterized to represent the mean and range of variability contrast medium propagation across a subset of patients. In order to do so, a particular XCAT model can be conditioned to represent the contrast material propagation that matches a mean across the represented patient group or individual patients within that group.

Confronting the challenge of modeling human body systems such as the cardiovascular system involves not only applying accurate mathematical models but also deploying the proper number of parameters governing the model. As the number of compartments in our PBPK model increases, the number of input parameters increases correspondingly. For this specific application of the PBPK model and level of accuracy that we seek, some model parameters, for instance permeability and membrane surface area, may not require precise determination. In this model, due to the lack of detailed anatomical information of permeability and surface area, they were considered as a product unit (PS) and assumed to be related to the blood flow rate in the organ depending on the flow-limited (PSI Q<1) or diffusion-limited (PSI Q>1) nature of the organ.

Due to the lack of specific data regarding the volumes of the extracellular (EC) and intracellular (IC) body fluids, it is assumed that the ratio of the EC and IC fluid volumes is fixed across all the organs and equals to the total ratio (0.83) calculated for the whole body. In reality, this ratio varies from patient to patient. This assumption was made since there is insufficient information for specific patients. This assumption may be revised based on the inherent vascular structure, further considering organ tissue heterogeneities. With the expanding use of multi-detector CT and the increasing clinical applications of CT angiography, a saline flush or chaser is now widely utilized with CT imaging to improve the efficiency of contrast medium administration. The saline injection can be modeled as a bi-phasic shape injection function in which the second phase is the contrast material pushed out of the veins in the arm into the central venous circulation. However, it has been shown that for a given amount of contrast media followed by the injection of a saline flush, results in a slight increase (5%-10%) in peak arterial enhancement, and the time-to-peak of enhancement. Also note that the contrast material arrival time or contrast enhancement magnitude of the first round of circulation is not affected by a saline flush. Another limitation of the implemented PBPK model is its validation against clinical mean liver and aorta data from two small groups of adult patients.

The majority of medical imaging systems take advantage of contrast medium administration in terms of better image quality, the effect of which has been ignored in previous optimization studies. Methods in accordance with embodiments of the present disclosure is provided to incorporate the dynamics of contrast material into computational anthropomorphic phantoms to create 5D XCAT models. The method may incorporate a physiologically based pharmacokinetics model and the anatomical parameters of the XCAT phantoms including height, weight, and organ volumes. Modeling contrast material propagation through multi-compartmental flow analysis offers validated contrast dynamics in a population of patients, enabling systematic optimization of CT in terms of image quality, radiation dose, and contrast load.

In an example study, radiation dose variation was quantified as a function of time due to the contrast medium administration in multiphase liver CT scan across a library of 5D XCAT models. In this study, the dose estimation was performed on a library of adult extended cardiac-torso (5D XCAT) models (35 male, 23 female, mean age 51.5 years, mean weight 80.2 kg). To generate the 5D XCAT patient models, a method in accordance with the present disclosure incorporates the dynamics of contrast-medium propagation into our 4D XCAT anthropomorphic phantoms. The models were created based on patient-specific iodine concentration-time results from our computational contrast medium propagation model for different intravenous injection protocols. Such that each organ in a patient model subjected to a specific injection protocol was assigned to its own unique contrast medium time-concentration curve. The radiation dose to individual organs in the models was estimated from a four-phase (pre-contrast, arterial phase, portal venous phase, and delayed phase) liver CT examination modeled via a validated Monte Carlo simulation software package (PENELOPE). For each scan time point after the injection, 80 million photons were initiated and tracked through the phantoms. Finally, the dose to the liver was tallied from the deposited energy. The liver CT scan simulation results from 5D XCAT models subjected to a commonly used injection protocol (120 mL of 350 mgI/mL contrast medium at 4 mL/s) indicated up to 10%, 33%, and 24% increase in radiation dose delivered to the liver for arterial phase (to 9.4 mGy), portal venous phase (to 11.4 mGy), and delay phase (to 10.6 mGy), respectively. Administration of contrast medium in enhanced CT scan not only remarkably affects the CT image quality (thus the reason for its use), but also notably increases the radiation dose. Particularly, multiple acquisitions in several enhanced CT protocols accentuate the radiation dose as a critical objective in optimization of the protocols. The relationship between radiation dose and injected contrast medium as a function of time studied in this work allows optimization of contrast administration for vulnerable individuals. The study aimed to provide a methodology to incorporate the contrast agent propagation in XCAT models, thus building toward an opportunity to optimize radiation dose and injection protocol in concert.

In another example study, an assessment was made of the effect of contrast medium on radiation dose as a function of time via Monte Carlo simulation from the liver CT scan across a library of 5D XCAT models. In this study, a validated Monte Carlo simulation package (PENELOPE) was employed to model a CT system (LightSpeed 64 VCT, GE Healthcare). The radiation dose was estimated from a common abdomen CT examination. The dose estimation was performed on a library of adult extended cardiac-torso (5D XCAT) phantoms (35 male, 23 female, mean age 51.5 years, mean weight 80.2 kg). The 5D XCAT models were created based on patient-specific iodine concentration-time results from our computational contrast medium propagation model for different intravenous injection protocols. To enable a dynamic estimation of radiation dose, each organ in the model was assigned to its own time-concentration curve via the PENELOPE package, material.exe. Using the Monte Carlo, for each scan time point after the injection, 80 million photons were initiated and tracked through the phantoms. Finally, the dose to the liver was tallied from the deposited energy. Monte Carlo simulation results of radiation dose delivered to the liver from the XCAT models indicated up to 30% increase in dose for different time after the administration of contrast medium. The contrast enhancement is employed in over 60% of imaging modalities, which not only remarkably affects the CT image quality, but also increases the radiation dose by as much as 70%. The post-injection multiple acquisition in several enhanced CT protocols, makes the radiation dose increment through the use of contrast medium, an inevitable factor in optimization of these protocols. The relationship between radiation dose and injected contrast medium as a function of time studied in this work allows optimization of contrast administration for vulnerable individuals.

In another example study, a method in accordance with the present disclosure incorporated the dynamics of blood flow and thus the distribution of contrast medium into the anthropomorphic phantom to generate 4D patient models for multimodality imaging studies. A compartmental model of blood circulation network within the body was embodied into an extended cardiac-torso (XCAT) patient model. To do so, a computational physiologic model of the human cardiovascular system was developed which includes a series of compartments representing heart, vessels, and organs. Patient-specific cardiac output and blood volume were used as inputs depending on the weight, height, age, and sex of the patient's model. For a given injection protocol and given XCAT model, the contrast medium transmission within the body is described by a series of mass balance differential equations, the solutions to which provides the contrast enhancement-time curves for each organ; thereby defining the tissue materials including the contrast medium within our XCAT model. A library of time-dependent organ materials was defined in the PENELOPE package, material.exe. Each organ in each voxelized 3D XCAT phantom was assigned to a corresponding time-varying material to create the 4D XCAT phantoms in which the fourth dimension is time. The model effectively predicts the time-varying concentration behavior of various contrast medium administration in each organ for different patient models. The contrast enhanced XCAT patient models was developed based on the concentration of iodine as function of time after injection. Majority of medical imaging systems take advantage of contrast medium administration in terms of better image quality, the effect of which was ignored in previous optimization studies. The study not only enables a comprehensive optimization of contrast administration both in terms of image quality and radiation dose, but can be easily developed into different modalities such as CT, MRI, and ultrasound.

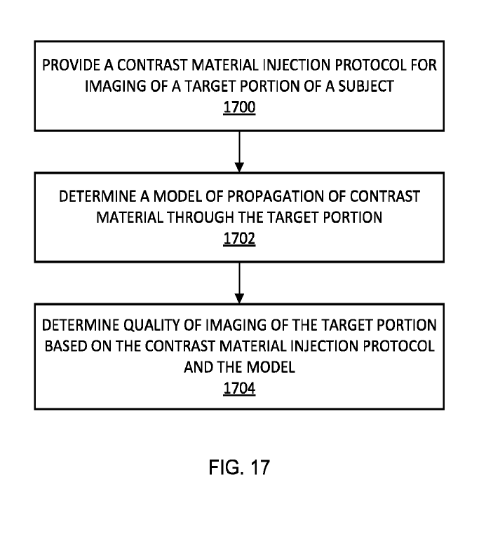

FIG. 17 illustrates a method of an example method for determining image quality in accordance with embodiments of the present disclosure. The method may be implemented by the system 100 shown in FIG. 1 or any other suitable system. Particularly, the method may be implemented by the computing device 113 shown in FIG. 1, and the results of the method may be applied to the system 100.

Referring to FIG. 17, the method includes providing 1702 a contrast material injection protocol for imaging of a target portion of a subject. A contrast material injection protocol may be a protocol that identifies the shape of an injection bolus by specifying one or more parameters of injection. Example injection parameters includes, but are not limited to, concentration, volume, rate, duration of injection, time-rate injection functions, and the like. An example injection for abdominal scan is injection of 125 mL of contrast medium, with concentration of 320 mg of iodine per milliliter at 4 mL/sec.

A subject may be a person or any other object for imaging. A target portion of a subject may be, for example, an organ, vessel, and/or other anatomical structure of the subject.

The method of FIG. 17 includes determining 1704 a model of propagation of contrast material through the target portion. Propagation of contrast material through a target may be modeled by use of a PBPK model. A PBPK model in general is a mathematical modeling technique for predicting the distribution, metabolism, and excretion (ADME) of synthetic or natural chemical substances in humans and other animal species. The compartmental model of propagation of contrast material disclosed herein is an instance of PBPK model which models the distributions of contrast material within the human body.

The method of FIG. 17 includes determining 1706 quality of imaging of the target portion based on the contrast material injection protocol and the model. Contrast material is administered to the body to help differentiate targeted areas of the body from background. By enhancing the image of specific organs, blood vessels or tissues, contrast materials help physicians diagnose medical conditions. Given a contrast administration method, the model can be used to determine how the contrast will be enhanced in the exam, thus predicting the expected quality from the examination. For example, when a 5 year old with a specific cardiovascular profile in being imaged, the model can predict the level of enhancement as a function of time. A quality of an image may be indicated by, for example, an image contrast, an image contrast-to-noise ratio, indices of detectability, indices of quantification, classification, or estimation and the like, and combinations thereof.

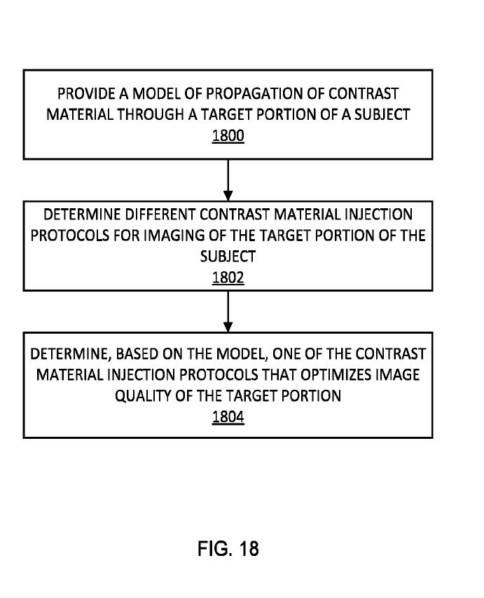

FIG. 18 illustrates a method of an example method for determining a contrast material injection protocol that optimizes image quality of a target portion of a subject in accordance with embodiments of the present disclosure. The method may be implemented by the system 100 shown in FIG. 1 or any other suitable system. Particularly, the method may be implemented by the computing device 113 shown in FIG. 1, and the results of the method may be applied to the system 100.

Referring to FIG. 18, the method includes providing 1800 a model of propagation of contrast material through a target portion of a subject. The model may be a PBPK model.

The method of FIG. 18 includes determining 1802 different contrast material injection protocols for imaging of the target portion of the subject targeting specific quality levels. A quality of an image may be indicated by, for example, an image contrast, an image contrast-to-noise ratio, indices of detectability, indices of quantification, classification, or estimation and the like, and combinations thereof. Example injection parameters includes, but are not limited to, concentration, volume, rate, duration of injection, the time-rate injection functions, and the like.

The method of FIG. 18 includes determining 1804, based on the model, one of the contrast material injection protocols that optimizes image quality of the target portion. If a particular enhancement level or image quality is desired, the methods described here can be used to determine contrast administration methods that produced that target. For example, if the specific level of contrast enhancement in the kidney is sought, based on the patient attributes, a contrast administration protocol can be defined (or chosen amongst multiple alternatives) to provide the closest possibility of achieving the target.

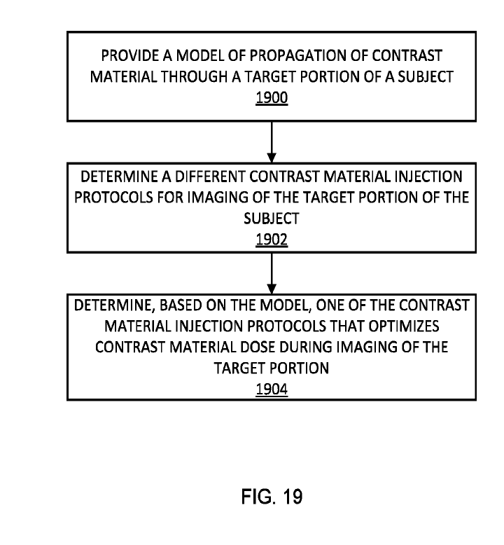

FIG. 19 illustrates a method of an example method for determining a contrast material injection protocol that optimizes contrast material dose during imaging of a target portion of a subject in accordance with embodiments of the present disclosure. The method may be implemented by the system 100 shown in FIG. 1 or any other suitable system. Particularly, the method may be implemented by the computing device 113 shown in FIG. 1, and the results of the method may be applied to the system 100.

Referring to FIG. 19, the method includes providing 1900 a model of propagation of contrast material through a target portion of a subject. The model may be a PBPK model.

The method of FIG. 19 includes determining 1902 different contrast material injection protocols for imaging of the target portion of the subject targeting specific contrast dose levels. Contrast dose level may be based on the total contrast burden to the whole body or to an organ of interest. Example injection parameters includes, but are not limited to, concentration, volume, rate, duration of injection, the time-rate injection functions, and the like.

The method of FIG. 19 includes determining 1904, based on the model, one of the contrast material injection protocols that optimizes contrast material dose during imaging of the target portion. If a particular contrast dose level should not be exceeded, the methods described here can be used to determine contrast administration methods that produced that target. For example, if the kidney contrast burden is not to exceed a specific level, based on the patient attributes, a contrast administration protocol can be defined (or chosen amongst multiple alternatives) to provide the closest possibility of achieving the target.

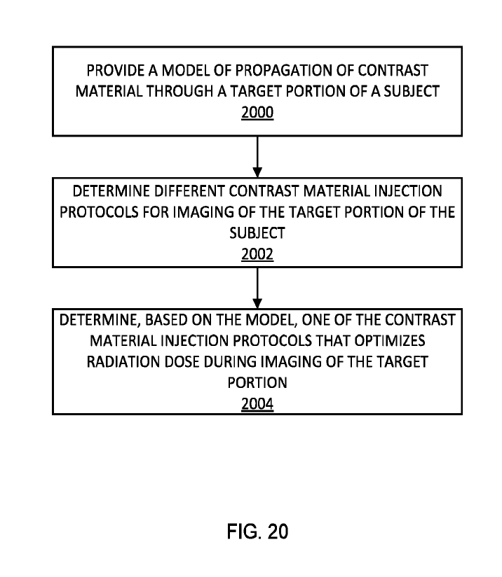

FIG. 20 illustrates a method of an example method for determining a contrast material injection protocol that optimizes radiation dose during imaging of a target portion of a subject in accordance with embodiments of the present disclosure. The method may be implemented by the system 100 shown in FIG. 1 or any other suitable system. Particularly, the method may be implemented by the computing device 113 shown in FIG. 1, and the results of the method may be applied to the system 100.

Referring to FIG. 20, the method includes providing 2000 a model of propagation of contrast material injection protocols for imaging of a target portion of a subject. The model may be a PBPK model.

The method of FIG. 20 includes determining 2002 different contrast material injection protocols for imaging of the target portion of the subject targeting specific radiation dose levels. Radiation dose level may be based on organ dose, dose indices (e.g., CT Dose Index or CTDI), effective dose, risk and risk indices, and the like, and combinations thereof. Example injection parameters includes, but are not limited to, concentration, volume, rate, duration of injection, the time-rate injection functions, and the like.

The method of FIG. 20 includes determining 2004, based on the model, one of the contrast material injection protocols that optimizes radiation dose during imaging of the target portion. If a particular radiation dose level is desired, the methods described here can be used to determine contrast administration methods that produced that target. For example, if the liver radiation dose not to exceed a specific level, based on the patient attributes, a contrast administration protocol can be defined (or chosen amongst multiple alternatives) to provide the closest possibility of achieving the target.

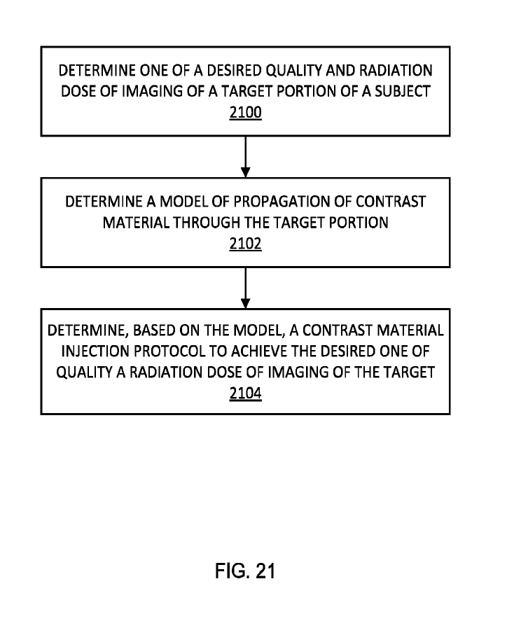

FIG. 21 illustrates a method of an example method for a contrast material injection protocol to achieve a desired quality or radiation dose of imaging of a target of a subject in accordance with embodiments of the present disclosure. The method may be implemented by the system 100 shown in FIG. 1 or any other suitable system. Particularly, the method may be implemented by the computing device 113 shown in FIG. 1, and the results of the method may be applied to the system 100.

Referring to FIG. 21, the method includes determining 2100 one of a desired quality and radiation dose of imaging of a target portion of a subject targeting specific radiation dose and image quality levels. Radiation dose level may be based on organ dose, dose indices (e.g., CT Dose Index or CTDI), effective dose, risk and risk indices, and the like, and combinations thereof. A quality of an image may be indicated by, for example, an image contrast, an image contrast-to-noise ratio, indices of detectability, indices of quantification, classification, or estimation and the like, and combinations thereof. Example injection parameters includes, but are not limited to, concentration, volume, rate, duration of injection, the time-rate injection functions, and the like.

The method of FIG. 21 includes determining 2102 a model of propagation of contrast material through the target portion. The model may be a PBPK model.

The method of FIG. 21 includes determining 2104, based on the model, a contrast material injection protocol to achieve the desired quality or radiation dose of imaging of the target. If a particular combination of radiation dose, contrast dose, and/or image quality level is desired, the methods described here can be used to determine contrast administration methods that produced that target. For example, if the liver CNR per radiation dose not to exceed a specific level, based on the patient attributes, a contrast administration protocol can be defined (or chosen amongst multiple alternatives) to provide the closest possibility of achieving the target.

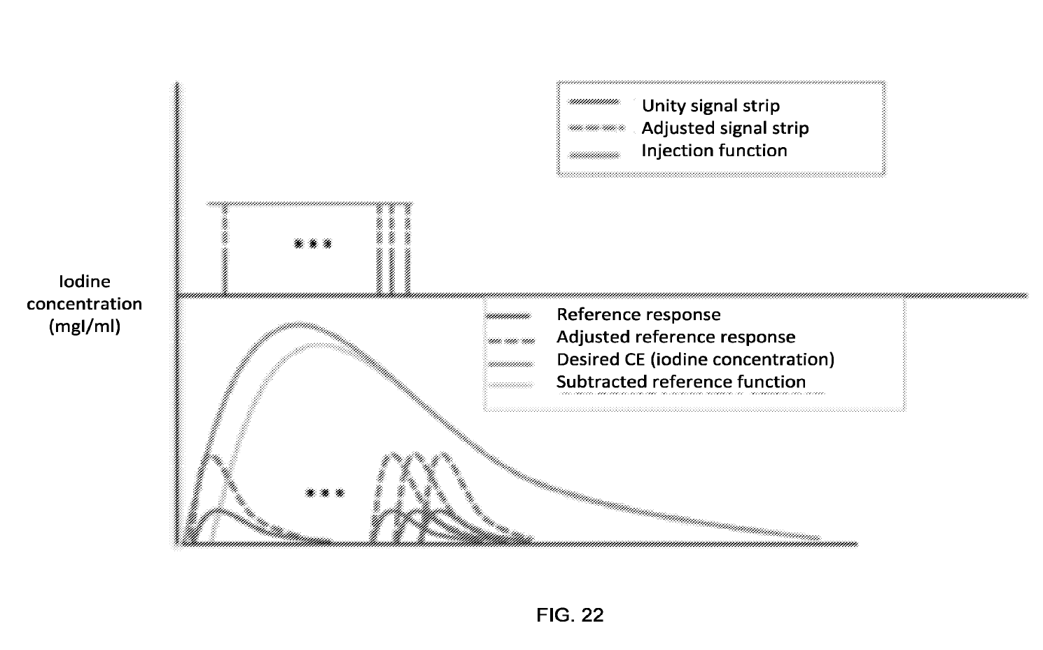

In accordance with embodiments, an iterative stripping technique may be utilized. In the iterative stripping technique, the injection IC function (signal) may be considered as deconvolution of the target IC (response). The iterative deconvolution technique assumes that the final target response is composed of the individual responses of a series of unit signals (strips). In this technique, the reference response may be calculated by applying a forward compartmental model as disclosed herein to the first unity signal strip (input function). The reference response amplitude may subsequently be adjusted to the magnitude of the target concentration value at the corresponding identical time point by multiplying it by an adjustment factor. This adjustment factor may also be applied to the unity signal strip. The adjusted reference response may be subtracted from the target concentration function to determine the response function without the contribution of the first signal. This step-by-step procedure may be iteratively repeated through a series of signal strips at multiple time points and subtracted reference functions. The iterative process was stopped when the remainder of the subtracted function reached a negative value. Finally, the injection function may be created by converting the estimated continuous function into a rectangular function as depicted in FIG. 22. Similar to the first method, the width of the rectangular function was calculated by dividing the area under the curve by the estimated continuous function plateau. By using analytical inverse and iterative stripping methods as disclosed herein, the injection functions can be predicted from the targeted organ enhancement functions across all XCAT models. Applying the forward compartmental odel on IFpredict, the predicted organ enhancement (OEpredict) curves may be calculated. For both methods, the accuracy of the predicted organ enhancement results were evaluated and compared across different protocols. The evaluation steps may be repeated across all the XCAT models described herein.

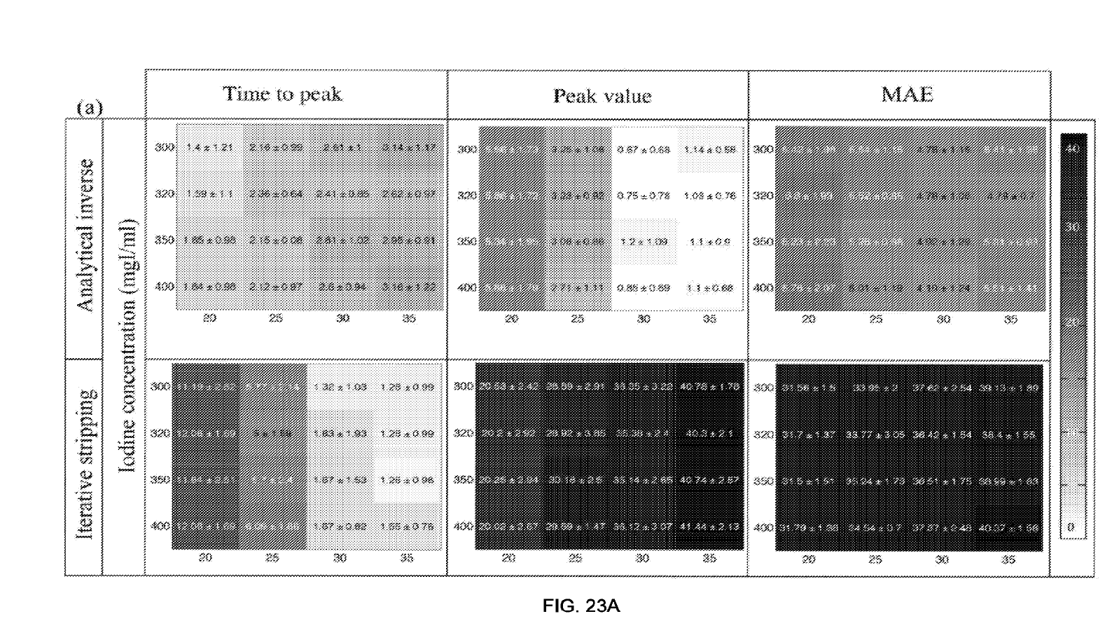

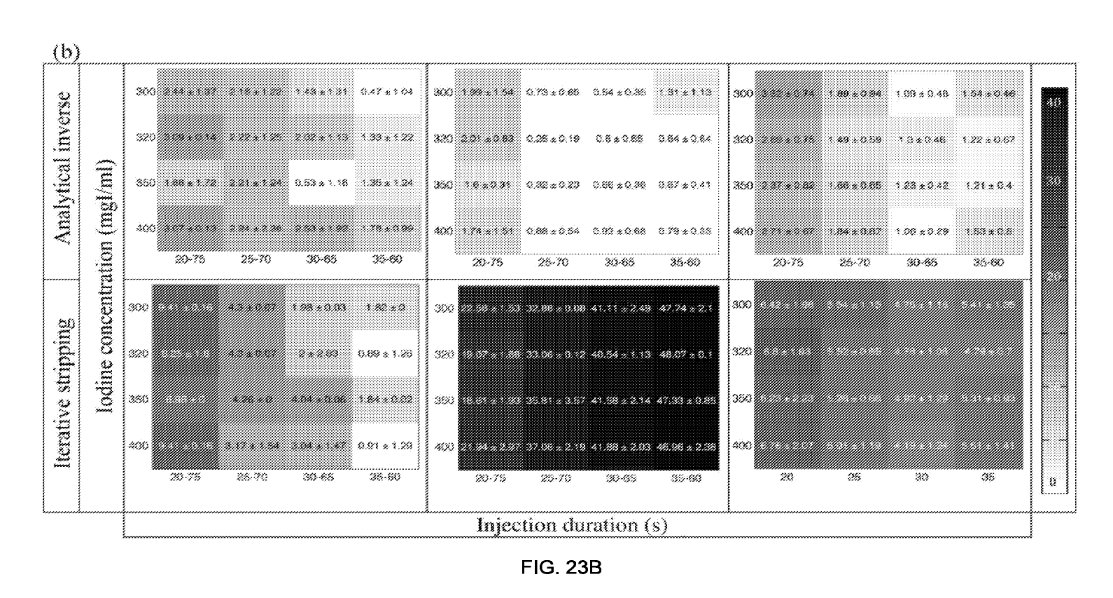

FIGS. 23A and 23B show an error map generated using the time-to-peak, peak value, and MAEs calculated for the two methods across uniphasic (See (a)) in FIG. 23A and biphasic (See (b) in FIG. 23B) injections. Each concentration-duration combination cell in the error blocks reports the averaged error computed over 58 patient models, i.e., the number represents the mean error+/−the standard deviation across all the models.

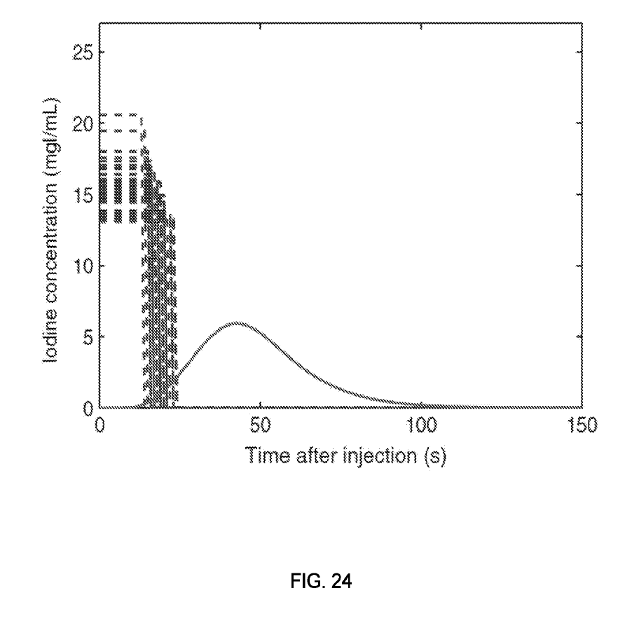

It is noted that the lack of patient-specific (e.g., size, gender, anatomy, and physiology) contrast material administration techniques can result in enhancement inconsistency across different patients. Applying the method developed in accordance with the present disclosure, required patient-specific function based on desired contrast enhancement may be designed by use of adjustable patient-specific parameters to provide a more consistent method. As evidence, applying the disclosed methods, the required injection function was calculated to achieve a given unique enhancement in the kidneys across a library of 58 XCAT models. FIG. 24 demonstrates that the injection duration and IC level can vary up to 66% and 76% across the anatomically variable XCAT patients, respectively.

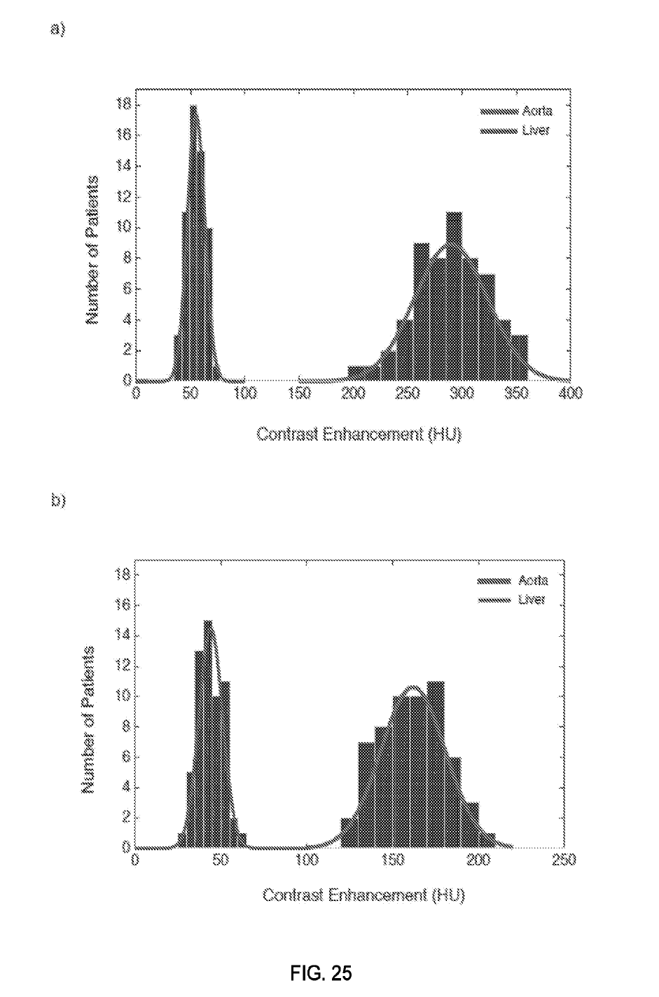

FIG. 25 illustrates graphs showing distribution of peak value of simulated arterial and hepatic contrast enhancement curves from 58 XCAT models for (a) uni-phasic and (b) bi-phasic injection protocols.

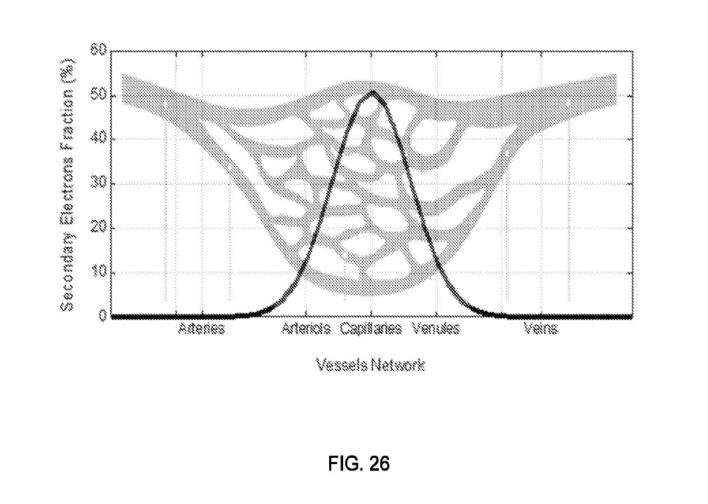

FIG. 26 depicts “tissue contribution” of radiation dose increase due to the presence of iodine in different components of vessels network. The distribution was approximated by probability of the secondary electrons which generated inside the iodine and deposited outside the iodine using a Monte Carlo simulation of a simplified organ model.

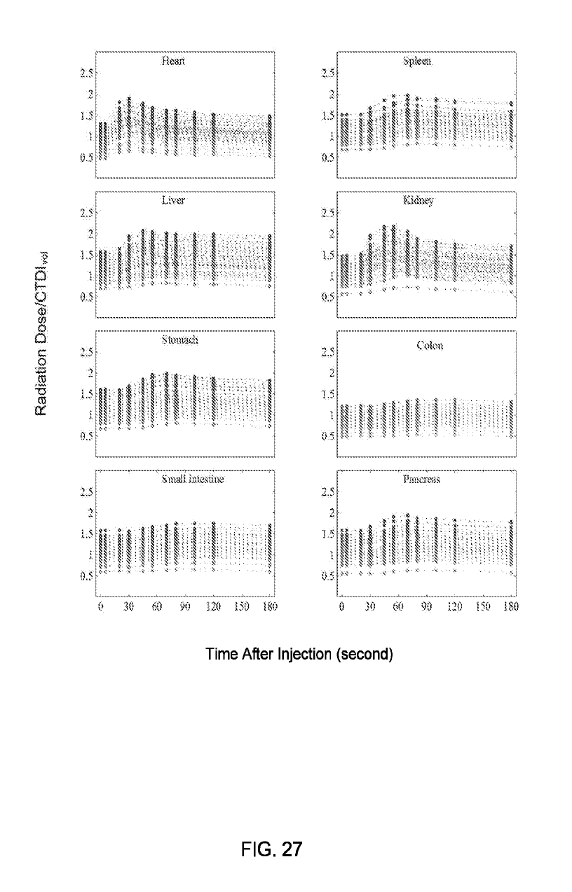

FIG. 27 depicts Monte Carlo simulation of the organ dose to the heart, spleen, liver, kidneys, stomach, colon, small intestine, and pancreas as a function of time across the XCAT models undergoing a contrast-enhanced abdomen CT scan. Each curve is representing an XCAT model. The organ doses are normalized by CTDIᵥₒₗ.

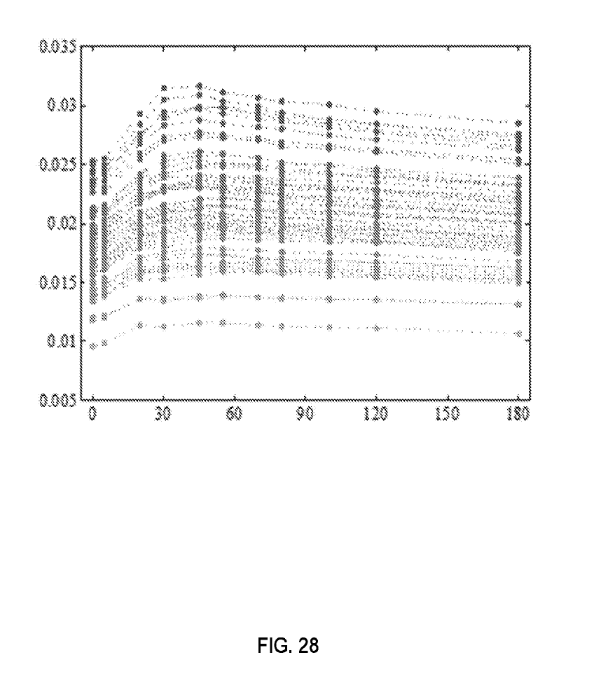

FIG. 28 shows calculated effective dose as function of time across XCAT models undergoing an abdomen contrast enhanced CT scan. The effective doses are normalized by DLP.

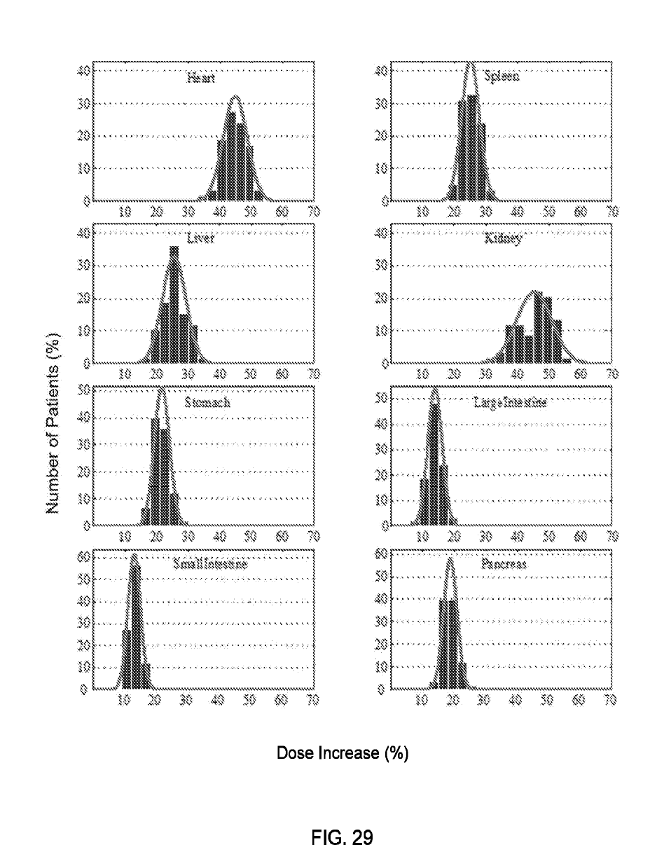

FIG. 29 shows distribution of the maximum dose increment (in percentage) in the heart, spleen, liver, kidney, stomach, colon, small intestine, and pancreas due to the administration of contrast medium across the XCAT models.

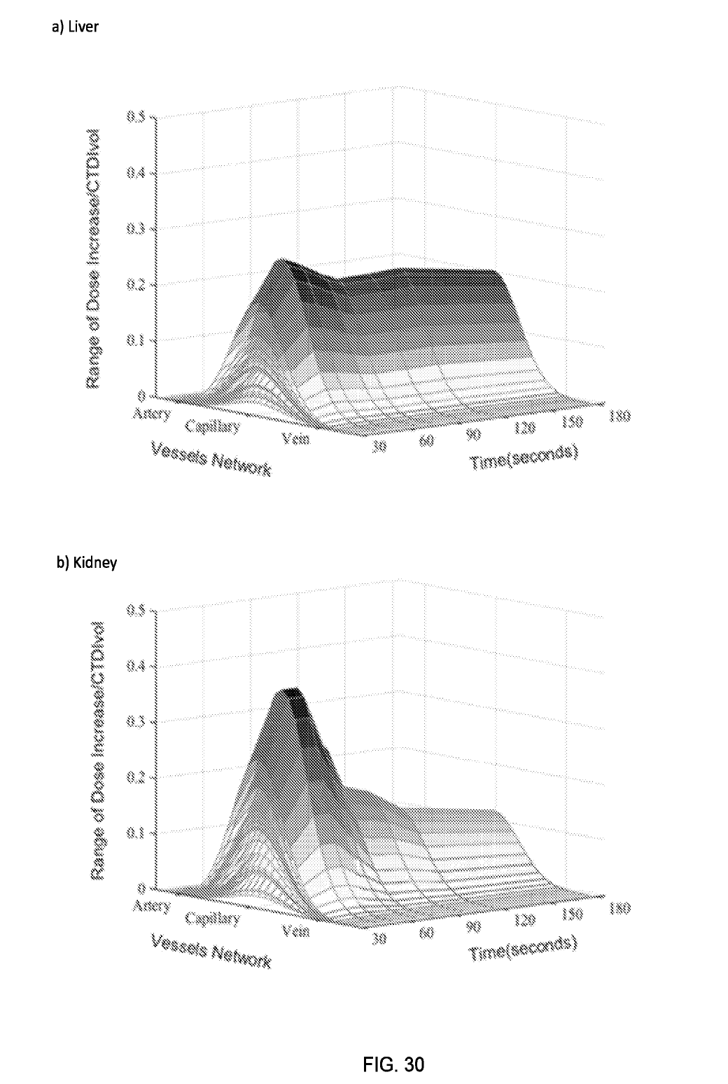

FIG. 30 shows potential range of radiation dose alteration for a) liver and b) kidney. The impact of iodine on radiation dose increase varies as the iodinated blood circulates through the vascular system. A normal distribution was assumed for the impact of iodine on dose increase as the blood enters the arteries and flows through the capillaries in which it experiences the maximum proximity to the tissue cells and washes out subsequently via the veins.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

While various embodiments of the present disclosure have been illustrated and described, it will be clear that the present disclosure is not limited to these embodiments only. Numerous modifications, changes, variations, substitutions, and equivalents will be apparent to those skilled in the art, without departing from the spirit and scope of the present disclosure. The singular forms “a,” “an,” and “the” include plural referents unless the context clearly dictates otherwise. Unless otherwise indicated, all numbers expressing quantities of distance, frequencies, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

1-46. (canceled)

47. A system comprising:
at least one processor and memory configured to:
provide a contrast material injection protocol for imaging of a target portion of a subject;
determine a model of propagation of contrast material through the target portion; and
determine quality of imaging of the target portion based on the contrast material injection protocol and the model.

48. The system of claim 47, wherein the imaging is computed tomography (CT) imaging and magnetic resonance imaging (MRI).

49. The system of claim 47, wherein the target portion of a subject comprises at least one of an organ, vessel and anatomical structure of the subject.

50. The system of claim 47, wherein the contrast material injection protocol defines at least one of timing, concentration, rate of injection, and injection timing profile for injecting contrast material into the subject.

51. The system of claim 47, wherein the determining a model of propagation of contrast material comprises determining one or more attributes of the subject.

52. The system of claim 51, wherein the determining one or more attributes of the subject comprises determining one of a height of the subject, a gender of the subject, an age of the subject, a weight of the subject, a cardiac output of the subject, a blood volume of portion of the subject, and metabolic profile of the subject.

53. The system of claim 51, wherein the determining one or more attributes of a subject comprises determining tracked contrast enhancements in the target portion of the subject.

54. The system of claim 47, wherein the determining quality of imaging comprises determining one of an image contrast, an image contrast-to-noise ratio, and indices of detectability or classification.

55. The system of claim 47, wherein the model of propagation of contrast material is a pharmacokinetic (PBPK) model.

56. The system of claim 55, wherein the PBPK model includes one of serial and parallel compartments of different volume.

57. The system of claim 55, wherein the PBPK model includes one of input and output flow characteristics for different organs and tissues.

58. The system of claim 47, wherein the model of propagation of contrast material relates one or more attributes of the subject to the contrast material injection protocol.

59. A system comprising:
at least one processor and memory configured to:
provide a model of propagation of contrast material through a target portion of a subject;
determine a plurality of different contrast material injection protocols for imaging of the target portion of the subject; and
determine, based on the model, one of the contrast material injection protocols that optimizes image quality of the target portion.

60. The system of claim 59, wherein the imaging is one of computed tomography (CT) imaging and magnetic resonance imaging (MRI).

61. The system of claim 59, wherein the target portion of a subject comprises at least one of an organ, vessel and anatomical structure of the subject.

62. The system of claim 59, wherein the model of propagation of contrast material is a pharmacokinetic (PBPK) model.

63. The system of claim 62, wherein the PBPK model includes one of serial and parallel compartments of different volume.

64. The system of claim 62, wherein the PBPK model includes one of input and output flow characteristics for different organs and tissues.

65. The system of claim 59, wherein the model of propagation of contrast material relates one or more attributes of the subject to the contrast material injection protocol.

66. A system comprising:
at least one processor and memory configured to:
provide a model of propagation of contrast material through a target portion of a subject;
determine a plurality of different contrast material injection protocols for imaging of the target portion of the subject; and
determine, based on the model, one of the contrast material injection protocols that optimizes contrast material dose during imaging of the target portion.

67. The system of claim 66, wherein the imaging is one of computed tomography (CT) imaging and magnetic resonance imaging (MRI).

68. The system of claim 66, wherein the target portion of a subject comprises at least one of an organ, vessel and anatomical structure of the subject.

69. The system of claim 66, wherein the model of propagation of contrast material is a pharmacokinetic (PBPK) model.

70. The system of claim 69, wherein the PBPK model includes one of serial and parallel compartments of different volume.

71. The system of claim 69, wherein the PBPK model includes one of input and output flow characteristics for different organs and tissues.

72. The system of claim 66, wherein the model of propagation of contrast material relates one or more attributes of the subject to the contrast material injection protocol.

73. A system comprising:
at least one processor and memory configured to:
provide a model of propagation of contrast material through a target portion of a subject;
determine a plurality of different contrast material injection protocols for imaging of the target portion of the subject; and
determine, based on the model, one of the contrast material injection protocols that optimizes radiation dose during imaging of the target portion.

74. The system of claim 73, wherein the imaging is one of computed tomography (CT) imaging and magnetic resonance imaging (MRI).

75. The system of claim 73, wherein the target portion of a subject comprises at least one of an organ, vessel and anatomical structure of the subject.

76. The system of claim 73, wherein the model of propagation of contrast material is a pharmacokinetic (PBPK) model.

77. The system of claim 76, wherein the PBPK model includes one of serial and parallel compartments of different volume.

78. The system of claim 76, wherein the PBPK model includes one of input and output flow characteristics for different organs and tissues.

79. The system of claim 73, wherein the model of propagation of contrast material relates one or more attributes of the subject to the contrast material injection protocol.

80. A system comprising:
at least one processor and memory configured to:
determine one of a desired quality and radiation dose of imaging of a target portion of a subject;
determine a model of propagation of contrast material through the target portion; and
determine, based on the model, a contrast material injection protocol to achieve the desired one of quality and radiation dose of imaging of the target.

81. The system of claim 80, wherein the imaging is one of computed tomography (CT) imaging and magnetic resonance imaging (MRI).

82. The system of claim 80, wherein the target portion of a subject comprises at least one of an organ, vessel and anatomical structure of the subject.

83. The system of claim 80, wherein determining a model of propagation of contrast material comprises determining one or more attributes of the subject.

84. The system of claim 83, wherein determining one or more attributes of the subject comprises determining one or more attributes of the subject comprises determining one of a height of the subject, a gender of the subject, an age of the subject, a weight of the subject, a cardiac output of the subject, a blood volume of portion of the subject, and the metabolic profile of the subject.

85. The system of claim 83, wherein determining one or more attributes of a subject comprises determining tracked contrast enhancements in the target portion of the subject

86. The system of claim 80, wherein determining a contrast material injection protocol comprises determining one of a volume of contrast material to inject, an injection rate, an injection magnitude, a concentration, an injection bolus, a saline chaser, and a factor governing one of magnitude and timing of the administration of the contrast material.

87. The system of claim 80, further comprising determining scan parameters for CT imaging of the target based on the model.

88. The system of claim 87, wherein determining scan parameters comprises determining one of a time of scan, photon energy spectrum, pitch, rotation time, table speed, and reconstruction parameters.

89. The system of claim 80, wherein the model of propagation of contrast material is a pharmacokinetic (PBPK) model.

90. The system of claim 89, wherein the PBPK model includes one of serial and parallel compartments of different volume.

91. The system of claim 89, wherein the PBPK model includes one of input and output flow characteristics for different organs and tissues.

92. The system of claim 80, wherein the model of propagation of contrast material relates one or more attributes of the subject to the contrast material injection protocol.
